(19) United States
(12) Patent Application Publication
(10) Pub. No.: US 2006/0134062 A1
(43) Pub. Date: Jun. 22, 2006

(54) Polymeric boronic acid derivatives as lipase inhibitors

(76) Inventors: Chad Cori Huval, Somerville, MA (US); Xinhua Li, Newton, MA (US); Stephen Randall Holmes-Farley, Arlington, MA (US); Pradeep K. Dhal, Westford, MA (US)

Correspondence Address: FISH & NEAVE IP GROUP;ROPES & GRAY LLP, ONE INTERNATIONAL PLACE, BOSTON, MA, 02110-2624, US

(21) Appl. No.: 10/535,639

(22) PCT Filed: Nov. 19, 2003
(86) PCT No.: PCT/US03/36861

§ 371 Date: Dec. 21, 2005

Related U.S. Application Data
(60) Provisional application No. 60/427,518, filed on Nov. 19, 2002.

(51) Int. Cl. A61K 31/785

(52) U.S. Cl. 424/78.27; 424/78.3

(57) ABSTRACT

Human obesity is a health problem affecting a significant proportion of the American population. Numerous methods of treating obesity have been developed, but all have serious drawbacks. The present invention discloses a novel class of polymers with either a sulfur atom or an electron withdrawing group between a polymer backbone and a pendant aryl boronic acid group. Polymers having such an electron withdrawing group have been found to be particularly effective in inhibiting lipase in vitro and in vivo. Methods of treating obesity and reducing absorption of fat are also disclosed.

### RELATED APPLICATION

This application claims the benefit of U.S. Provisional Application No. 60/427,518, filed on Nov. 19, 2002. The entire teachings of the above application are incorporated herein by reference.

### BACKGROUND OF THE INVENTION

Human obesity is a recognized health problem with approximately ninety-seven million people considered clinically overweight in the United States.

The accumulation or maintenance of body fat bears a direct relationship to caloric intake. Therefore, one of the most common methods for weight control to combat obesity is the use of relatively low-fat diets; that is, diets containing less fat than a “normal diet” or that amount usually consumed by the patient.

The presence of fats in a great many food sources greatly limits the food sources that can be used in a low-fat diet. Additionally, fats contribute to the flavor, appearance and physical characteristics of many foodstuffs. As such, the acceptability of low-fat diets and the maintenance of such diets are difficult.

Various chemical approaches have been proposed for controlling obesity. Anorectic agents such as dextroamphetamine, the combination of the non-amphetamine drugs phentermine and fenfluramine (Phen-Fen), and dexfenfluramine (Redux) alone, are associated with serious side effects. Indigestible materials such as olestra (OLEAN®), mineral oil or neopentyl esters (see U.S. Pat. No. 2,962,419) have been proposed as substitutes for dietary fat. Garcinia acid and derivatives thereof have been described as treating obesity by interfering with fatty acid synthesis. Swellable crosslinked vinyl pyridine resins have been described as appetite suppressants via the mechanism of providing non-nutritive bulk, as in U.S. Pat. No. 2,923,662. Surgical techniques such as temporary ileal bypass surgery are employed in extreme cases.

However, methods for treating obesity, such as those described above have serious shortcomings with controlled diet remaining the most prevalent technique for controlling obesity. As such, new methods for treating obesity are needed.

### SUMMARY OF THE INVENTION

It has now been found that polymers having an electron withdrawing group, such as a carbonyl group, para or meta relative to a pendant aryl boronic acid group, are particularly effective in inhibiting lipase in vitro (Example 61) and in vivo (Example 62) when there is a linker of adequate length connecting the aryl boronic acid moiety to the polymer. Other polymers having a hydrocarbylene moiety interrupted by a sulfur atom linking a boronic acid and the polymer backbone also have activity against lipase in vitro (Example 61) and in vivo (Example 62) and can be readily synthesized. Based on these discoveries, polymers with pendant boronic acid groups and appropriate groups linking the boronic acid group to the polymer are disclosed herein. Pharmaceutical compositions comprising these polymers and methods of treatment using these polymers are also disclosed.

In one embodiment, the present invention is a polymer substituted with at least one group represented by Structural Formula (I) or (II):

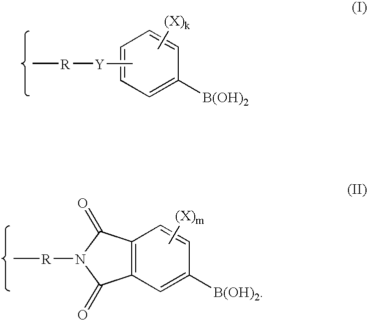

In Structural Formulas (I) and (II), R is a C6-C30 hydrocarbylene group optionally interrupted by one or more heteroatoms selected from the group consisting of NH, S, and O.

Each X is independently —H, a substituted or unsubstituted alkyl group, an electron withdrawing group, or an electron donating group meta to the boronic acid moiety.

Y is —C(O)Z-, -ZC(O)— or —S(CH₂)ₙ—.

Z is a bond, CH₂S, S, NH or O.

m is an integer from 0 to 3.

k is an integer from 0 to 4.

n is an integer from 0 to 5.

The present invention also includes a method for treating obesity in a mammal and a method for reducing absorption of fat in a mammal in need of treatment therefor. The methods comprise the step of orally administering to the mammal an effective amount of a polymer disclosed herein.

A pharmaceutical composition comprised of one or more polymers of the present invention, along with a carrier or diluent, is another aspect of the invention. The pharmaceutical composition can be used for therapy, such as in the treatment of a disorder described herein. Similarly, the invention provides for the use of a polymer disclosed herein as a medicament and for the use of a polymer disclosed herein in the manufacture of a medicament for the treatment of a disorder described herein.

Polymers disclosed herein are readily synthesized and highly effective in inhibiting lipase both in vivo and in vitro. As a result, the polymers are also effective in the treatment of obesity and many conditions or diseases associated with obesity. Additional advantages of polymers of the present invention include backbones that are non-degradable under physiological conditions. As a consequence, the polymers are substantially not absorbed by the gastrointestinal tract. As such, the polymers are expected to be non-toxic and non-antigenic.

### DETAILED DESCRIPTION OF THE INVENTION

Disclosed herein are novel polymers substituted with pendant aryl boronic acid groups and methods of use therefor. The polymers disclosed herein can, for example, be substituted with a group or groups represented by Structural Formula (I) or (II). Preferably, polymers of the present invention are substituted with at least one group represented by Structural Formula (III) or (IV): 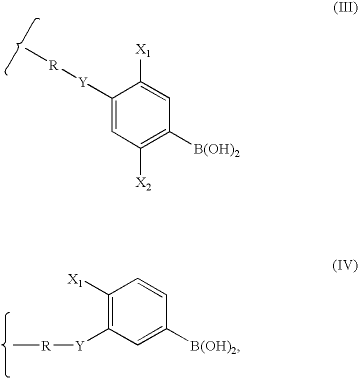
where X₁ and X₂ are each independently —H, a halogen, nitrile, ester or sulfone; and R and Y are as above.

In Structural Formulas (III) and (IV), Y is preferably —C(O)Z- or -ZC(O)—. More preferably, Y is -ZC(O)—. These and other formulas shown herein are meant to be read from left to right in the structures in which they are found. Thus, for example, when Y in Structural Formula (III) is —OC(O)—, the carbonyl carbon is bonded to the phenyl ring and the “non-carbonyl oxygen” is bonded to R. Even more preferably, Y is —OC(O)—, —SC(O)—, or —SCH₂C(O)— and R is a C6-C12 alkylene group. Preferred values of X in Structural Formulas (III) and (IV) are —H, —F, —CH₃, or —CH₂CH₃.

A specific example of a group represented by Structural Formula (III) is a group represented by Structural Formula (V): 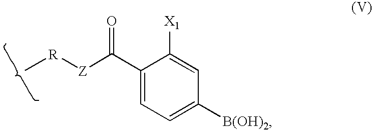
where R, X₁, and Z are as above.

Examples of groups represented by Structural Formula (V) include groups represented by Structural Formulas (VI), (VII), and (VIII): 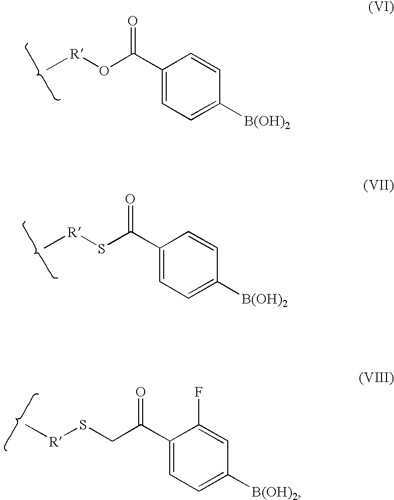
where R′ is a C6-C12 alkylene group.

Polymer substituted with groups represented by Structural Formulas (I)-(VIII) are advantageously substituted with at least. two such groups, such as at least ten such groups. For example, at least about 5% of the repeat units can be substituted with a group represented by one or more of Structural Formulas (I)-(VIII), at least about 10% of the repeat units can be substituted with a group represented by one or more of Structural Formulas (I)-(VIII), at least about 20% of the repeat units can be substituted with a group represented by one or more of Structural Formulas (I)-(VIII), at least about 30% of the repeat units can be substituted with a group represented by one or more of Structural Formulas (I)-(VIII), at least about 40% of the repeat units can be substituted with a group represented by one or more of Structural Formulas (I)-(VIII), at least about 50% of the repeat units can be substituted with a group represented by one or more of Structural Formulas (I)-(VIII), at least about 60% of the repeat units can be substituted with a group represented by one or more of Structural Formulas (I)-(VIII), at least about 70% of the repeat units can be substituted with a group represented by one or more of Structural Formulas (I)-(VIII), at least about 80% of the repeat units can be substituted with a group represented by one or more of Structural Formulas (I)-(VIII) or at least about 90% of the repeat units can be substituted with a group represented by one or more of Structural Formulas (I)-(VIII).

In another embodiment, the present invention is a polymer comprised of polymerized monomer units, wherein the monomer unit is represented by Structural Formula (IX), (X), or (XI):

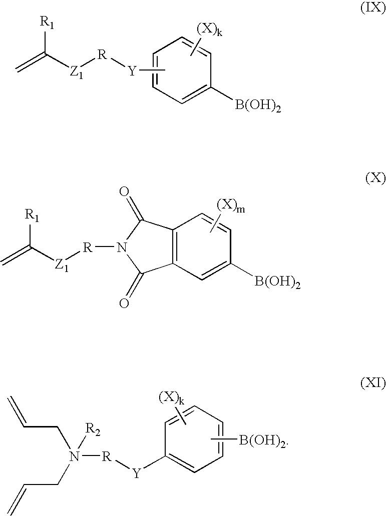

In Structural Formulas (IX), (X), and (XI), R is a C6-C30 hydrocarbylene group optionally interrupted by one or more heteroatoms selected from the group consisting of NH, S and O.

R₁ is —H or a lower alkyl group.

R₂ is —H, a lower alkyl group, or is absent.

Each X is independently —H, a substituted or unsubstituted alkyl group, or an electron withdrawing group.

Y is —C(O)Z-, -ZC(O)— or —S(CH₂)ₙ—.

Z is a bond, CH₂S, S, NH or O.

Z₁ is a bond, —C(O)NH—, —C(O)O—, —C₆H₄O—, or —C₆H₄NHC(O)—.

m is an integer from 0 to 3.

k is an integer from 0 to 4.

n is an integer from 0 to 5.

Preferred polymers are comprised of polymerized monomer units where the monomer unit is represented by Structural Formula (XII) or (XIII)

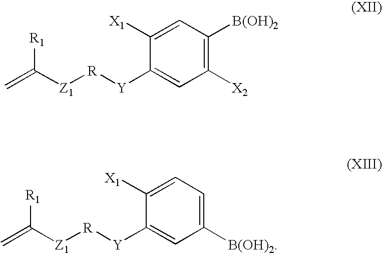

X₁ and X₂ are each independently —H, a halogen, nitrile, ester or sulfone.

R, R₁, Y and Z₁ are as defined above.

Typically, monomer units represented by Structural Formulas (XII) and (XIII) have one, two, three, four, five, or six of the following features: (1) R is a C6-C12 alkylene group; (2) R₁ is —H; (3) X₁ and (4) X₂ are each independently —H or —F; (5) Y is —OC(O)— or —SCH₂C(O)—; and (6) Z₁ is —C(O)O—. Preferably, the monomer units have feature (1), features (1) and (2), features (1), (2) and (3), features (1), (2), (3) and (4) or features (1), (2), (3), (4) and (5). More preferably, monomer units represented by Structural Formulas (XII) and (XIII) have all six of the features listed above.

In specific examples, polymers of the present invention are comprised of polymerized monomer units where the monomer unit is represented by Structural Formula (XIV), (XV), (XVI) or (XVII):

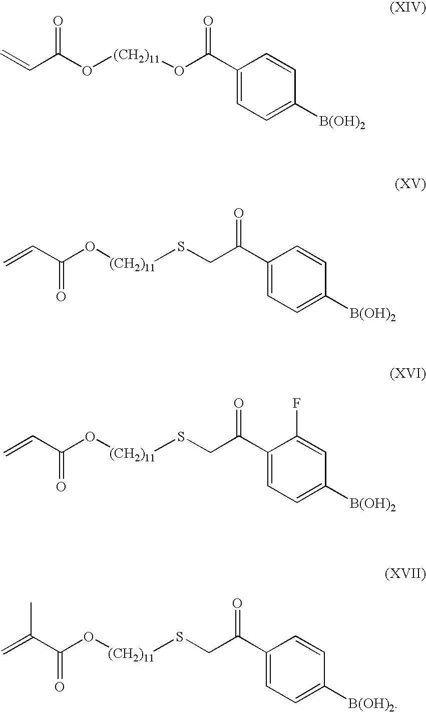

For polymers substituted with groups represented by Structural Formula (I) or (II) or polymers comprised of polymerized monomer units represented by Structural Formula (IX), (X), or (XI), each X is preferably independently —H, a halogen or nitrile. Groups such as —S(CH₂)ₙ—, —SCH₂C(O)—, and —SCH₂— are preferably oriented in the moiety linking the boronic acid group to the polymer backbone such that the sulfur atom is distant from the boronic acid group and closer to the polymer backbone.

Additional polymers for use in the present invention are comprised of polymerized monomer units where the monomer unit is represented by the following formulas: 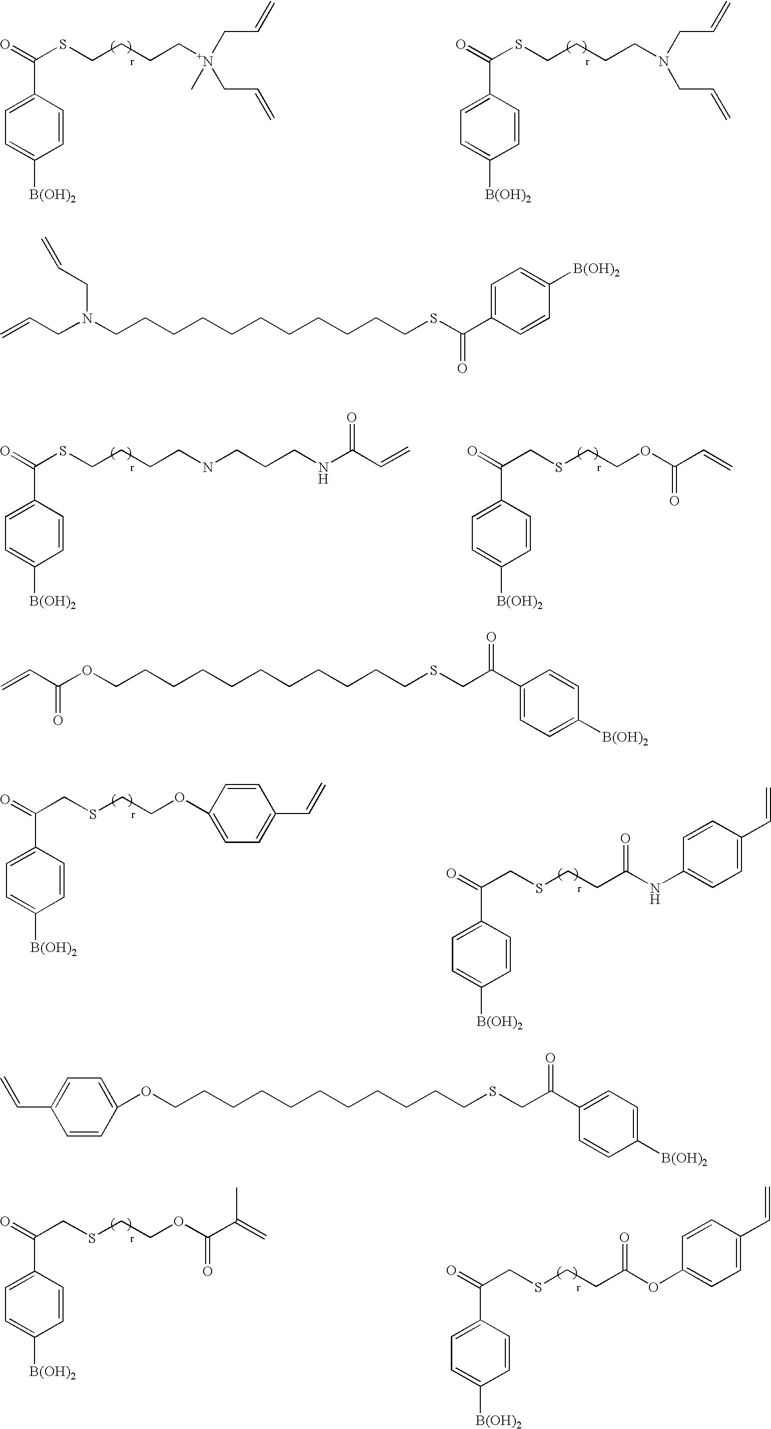 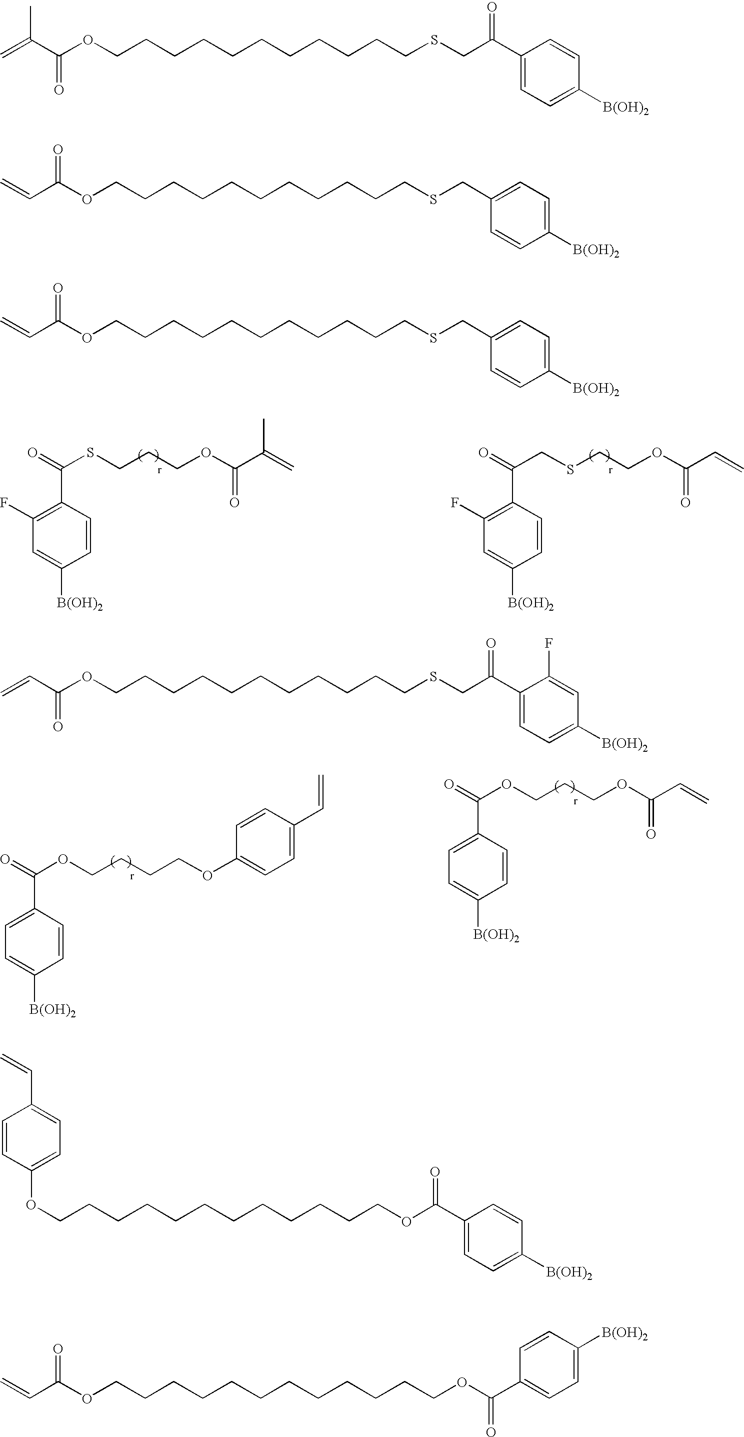 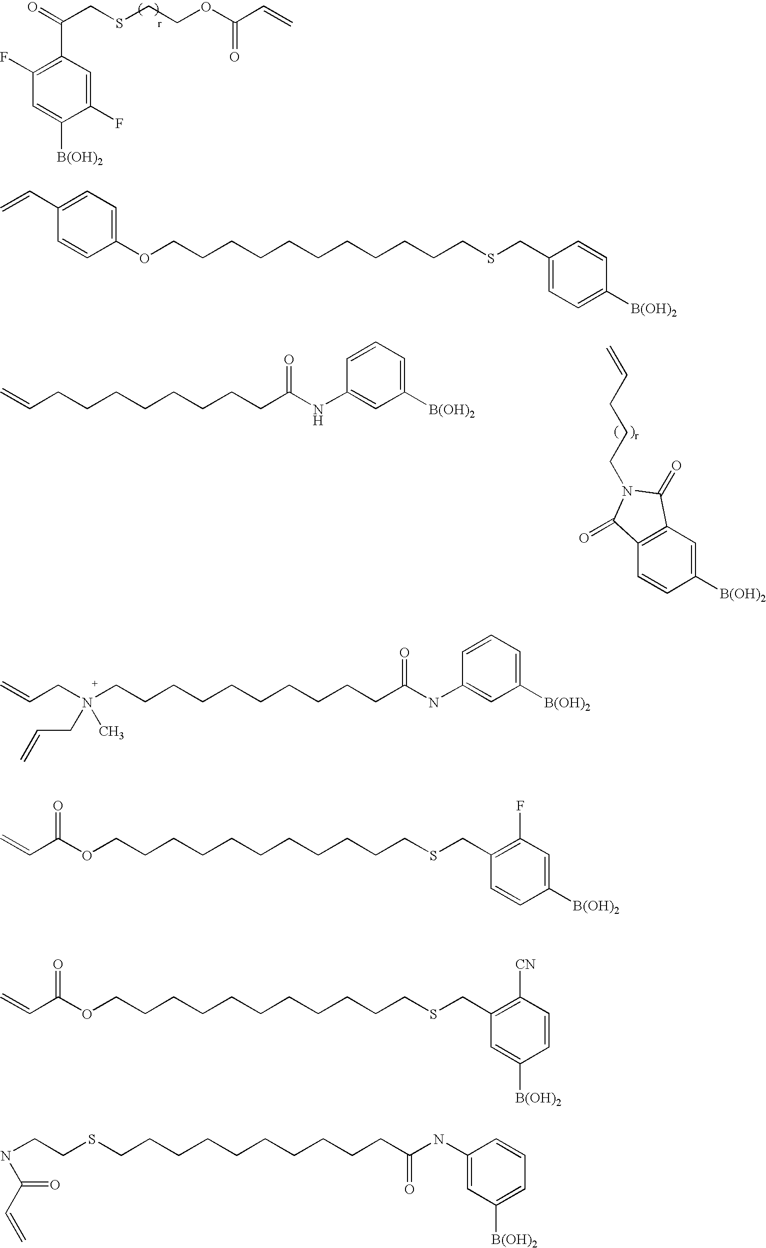 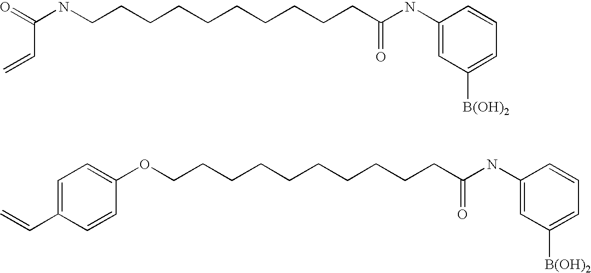
In the structures shown immediately above, “r” represents an integer from 0 to 10, such as from 0 to 8, for example 3 to 8.

Variables of polymers represented herein are typically chosen such that a polymer of the present invention has one or more of the following features: a) non-degradable under physiological conditions, b) adequate molecular weight to be non-absorbable, c) a hydrophobic spacer of appropriate length and flexibility to interact with an active site of lipase, d) a large number of boronic acid groups per polymer chain (e.g., to increase the effective concentration of boronic acid groups and lower the effective dose of polymer); e) the original specificity and activity of parent boronic acid is retained; and f) solubility in a triglyceride emulsion under physiological conditions. Typically, polymers of the present invention have hydrophilic backbone structures, while groups linking a boronic acid group with a backbone are hydrophobic or primarily hydrophobic in character.

Polymers of the present invention can be copolymers, i.e., comprise two or more different repeat units (monomers). One of these repeat units comprises one of the disclosed boronic acid containing groups or is one of the boronic acid containing polymerized monomers. A second repeat unit is a cationic, anionic, zwitterionic or neutral hydrophilic repeat unit or a hydrophobic repeat unit. A copolymer can have more than one cationic, anionic, zwitterionic or neutral hydrophilic repeat unit and more than one hydrophobic repeat unit. Copolymers can be prepared by direct polymerization of two or more monomers or by chemical modification of a reactive polymer. Preferably, the copolymer comprises an anionic repeat unit or a zwitterionic repeat unit.

Copolymers of the present invention can exist in a variety of forms. Suitable forms include block copolymers, graft copolymers, comb copolymers, star copolymers, dendrimers, hyperbranched copolymers, crosslinked hydrogels, random copolymers, gradient block copolymers, and alternate copolymers.

Especially preferred copolymers include poly{4-(14′-acryloxy-3′-thia-1′-keto)tetradecyl phenyl boronic acid-co-potassium 3-sulfopropyl acrylate)}, poly{4-(14′-methacryloxy-3-thia-1′-keto)tetradecyl phenyl boronic acid-co-sodium 4-styrene sulfonate}, poly{11-acryloxyundecyl(4-boronato)benzoate-co-sodium 2-acrylamido-2-methyl-1-propanesulfonate}, poly{4-(14′-acryloxy-3′-thia-1′-keto)tetradecyl phenyl boronic acid-co-sodium 2-acrylamido-2-methyl-1-propanesulfonate}, or poly{4-(14′-acryloxy-3-thia-1′-keto)tetradecyl phenyl boronic acid-co-sodium-4-styrene sulfonate}.

Examples of suitable cationic monomers have an ammonium group and include monomers represented by the following structures:

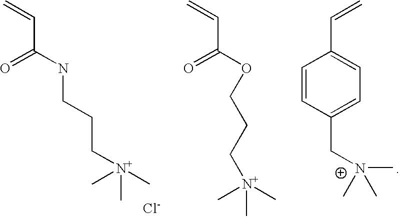

A neutral hydrophilic repeat unit can, for example, comprise a polyether sidechain, as shown below. Other examples of suitable neutral hydrophilic monomers include acrylamide monomers and monomers with alcohol-containing pendant groups, such as the monomers represented by the following structures:

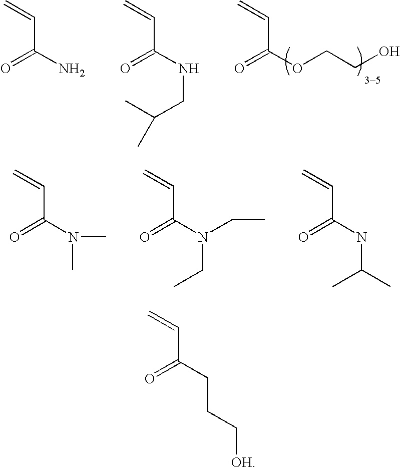

Negatively-charged monomers include those comprising a sulfonic acid moiety or a salt thereof, such as 2-acrylamido-2-methyl-1-propane sulfonic acid and salts thereof, styrene sulfonic acid and salts thereof, and 3-acrylatopropane sulfonic acid and salts thereof. Other examples of negatively-charged repeat units include those comprising a carboxylic acid or phosphoric acid moiety or a salt thereof, such as acrylic or maleic acid. Examples of suitable anionic monomers include monomers represented by the following structures:

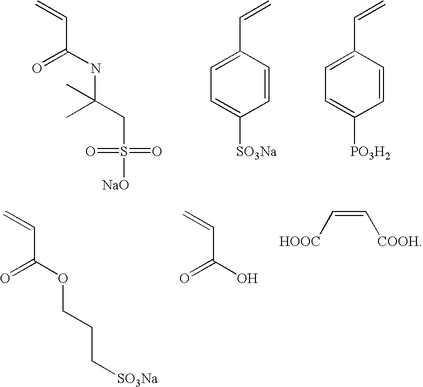

Zwitterionic monomers include those comprising a sulfonic acid moiety or a salt thereof. An example of a zwitterionic monomer is represented by the structure:

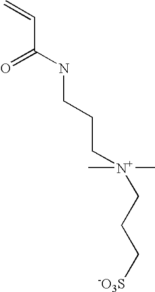

Although the polymer backbone is not believed to be critical (although some backbones may have more desirable properties), examples of polymer backbones that can be substituted with one or more pendant boronic acid groups include vinyl polymers such as a polyacrylate, alkylpolyacrylate, polyacrylamide, alkylpolyacrylamide, poly(allylalcohol), poly(vinylalcohol), poly(vinylamine), poly(allylamine), poly(diallylamine) or a substituted polystyrene backbone. Groups comprising aryl boronic acids are attached, for example, by ester linkages to carboxylate groups of a polyacrylate, by a covalent bond to the amide nitrogens of a polyacrylamide, by ether linkages to alcohols of a poly(vinylalcohol) or poly(allylalcohol), by a covalent bond to the amines of a poly(vinylamine,) a poly(allylamine) or a poly(diallylamine) or by a covalent bond to a substituent on the phenyl ring of a polystyrene. Polyacrylamide, polyacrylate, polystyrene 4-alcohol, polyethylene, poly(N-carboxy-4-aminostyrene), polydiallylamine are preferred polymers.

Additional suitable polymer backbones that can be substituted with one or more pendant boronic acid groups include substituted or poly-N-alkylvinylamine, poly-N-alkylallylamine, poly-N-alkyldiallylamine, polyalkylenimine, other polyamines, polyethers, polyamides, polyacrylic acids, polyalkylacrylates, polymethacrylic acids, polyalkylmethacrylates, polymethacrylamides, poly-N-alkylacrylamides, poly-N-alkylmethacrylamides, polyvinylnaphthalene, polyethylvinylbenzene, polyaminostyrene, polyvinylbiphenyl, polyvinylanisole, polyvinylimidazole, polyvinylpyridine, polydimethylaminomethylstyrene, polydiallylmethylammonium chloride, polytrimethylammonium ethyl methacrylate, polytrimethylammonium ethyl acrylate, and copolymers thereof.

Condensation polymers, which are formed from reactions in which a small molecule such as water is released, are also suitable polymer backbones. Examples of condensation polymers include polyamides, polyalkyleneimines and polyesters. A polyalkyleneimine can have amine or ammonium nitrogens in the backbone. A pendant group comprising a hydrocarbylene group and a boronic acid containing group can be connected to a polyalkyleneimine, for example, by the amine or ammonium nitrogens in the backbone or, alternatively, ammoniumalkyl (e.g., a trialkylammonium alkyl group) or hydroxylated alkyl groups (e.g., hydroxyethyl) bonded to nitrogen in the polymer backbone. For polyamides, a pendant group can be bonded to a carbon atom or an amide nitrogen in the polymer backbone. For polyesters, a pendant group can be bonded to a carbon atom in the backbone.

The polymer can be linear or crosslinked. Crosslinking can be performed by reacting the polymer with one or more crosslinking agents having two or more functional groups, such as electrophilic groups, which react with, for example, amine groups to form a covalent bond. Crosslinking in this case can occur, for example, via nucleophilic attack of the polymer amino groups on the electrophilic groups. This results in the formation of a bridging unit which links two or more amino nitrogen atoms from different polymer strands. Suitable crosslinking agents of this type include compounds having two or more groups selected from among acyl chloride, epoxide, and alkyl-X, wherein X is a suitable leaving group, such as a halo, tosyl or mesyl group. Examples of such compounds include, but are not limited to, epichlorohydrin, succinyl dichloride, acryloyl chloride, butanedioldiglycidyl ether, ethanedioldiglycidyl ether, pyromellitic dianhydride, and dihaloalkanes. These crosslinking agents are referred to herein as multifunctional crosslinking agents.

The polymer composition can also be crosslinked by including a multifunctional co-monomer as the crosslinking agent in the polymerization reaction mixture. A multifunctional co-monomer can be incorporated into two or more growing polymer chains, thereby crosslinking the chains. Suitable multifunctional co-monomers include, but are not limited to, diacrylates, triacrylates, and tetraacrylates, dimethacrylates, diacrylamides, and dimethacrylamides. Specific examples include ethylene glycol diacrylate, propylene glycol diacrylate, butylene glycol diacrylate, ethylene glycol dimethacrylate, butylene glycol dimethacrylate, methylene bis(methacrylamide), ethylene bis(acrylamide), ethylene bis(methacrylamide), ethylidene bis(acrylamide), ethylidene bis(methacrylamide), pentaerytiritol tetraacrylate, trimethylolpropane triacrylate, bisphenol A dimethacrylate, and bisphenol A diacrylate. Other suitable multifunctional monomers include polyvinylarenes, such as divinylbenzene.

When crosslinked, the amount of cross-linking agent is typically between about 0.01 and about 10 weight % based on the combined weight of crosslinking agent and monomers, with 0.1-3% being preferred. Typically, the amount of cross-linking agent that is reacted with the polymer, when the crosslinking agent is a multifunctional agent, is sufficient to cause between about 0.1 and 6% of the nucleophiles present on the monomer, for example, an amine to react with the crosslinking agent.

Also included in the present invention are pharmaceutically acceptable salts of the disclosed polymers. For example, polymers which have acid functional groups can also be present in the anionic, or conjugate base, form, in combination with a cation. Suitable cations include alkaline earth metal ions, such as sodium and potassium ions, alkaline earth ions, such as calcium and magnesium ions, and unsubstituted and substituted (primary, secondary, tertiary and quaternary) ammonium ions. Polymers which have basic groups such as amines can also be protonated and have a pharmaceutically acceptable counter anion, such as halides (Cl⁻ and Br⁻), CH₃OSO₃⁻, HSO₄⁻, SO₄²⁻, HCO₃⁻, CO₃²⁻, nitrate, hydroxide, persulfate, sulfite, acetate, formate, sulfate, phosphate, lactate, succinate, propionate, oxalate, butyrate, ascorbate, citrate, dihydrogen citrate, tartrate, taurocholate, glycocholate, cholate, hydrogen citrate, maleate, benzoate, folate, an amino acid derivative, a nucleotide, a lipid, or a phospholipid. Similarly, ammonium groups comprise a pharmaceutically acceptable counteranion. Boronic acid groups can react with anions such as sodium or potassium hydroxide, alkoxide or carboxylate to form a salt such as —B⁻(OH)₃Na⁺, —B⁻(OH)₃K⁺, —B⁻(OH)₂(OCH₃)Na⁺, —B⁻(OH)₂(OCH₃)K⁺, —B⁻(OH)₂(OCOCH₃)Na⁺, —B⁻(OH)₂(OCOCH₃)K⁺, and the like.

The polymers of the present invention are advantageously co-administered to a mammal together with a fat binding polymer. Fat binding polymers include those described in, for example, U.S. Pat. Nos. 6,030,953, 6,251,421, 6,352,692, 6,299,868, 6,267,952, 6,264,937, and 6,358,522, the contents of which are incorporated herein by reference. Examples of fat binding polymers include, for example, chitosan, carbophil, and water-soluble polysaccharides such as microcrystalline cellulose, methylcellulose, xanthan gum, psyllium seed, ispaghula husk, plantago ovata seeds, and karaya gum. Other suitable fat binding polymers have a positively-charged region, a hydrophobic region, or a region that is both positively-charged and hydrophobic, particularly those that are non-absorbable and have a non-hydrolyzable backbone.

Mammals include humans, companion animals (e.g., dogs, cats, and the like), farm animals (e.g., cows, sheep, pigs, horses, and the like) and laboratory animals (e.g., rats, mice, guinea pigs, and the like) in need of treatment for obesity or in need or treatment for reducing fat absorption.

A mammal in need of treatment for reducing fat absorption is a mammal suffering from one or more of the following conditions: obesity, Type II (non-insulin-dependent) diabetes mellitus, impaired glucose tolerance, hypertension, coronary thrombosis, stroke, lipid syndromes, hyperglycemia, hypertriglyceridemia, hyperlipidemia, sleep apnea, hiatal hemia, reflux esophagisitis, osteoarthritis, gout, cancers associated with weight gain, gallstones, kidney stones, pulmonary hypertension, infertility, cardiovascular disease, above normal weight, and above normal lipid levels; or where the subject would benefit from reduced platelet adhesiveness, weight loss after pregnancy, lowered lipid levels, lowered uric acid levels, or lowered oxalate levels.

The polymers of the present invention are suitable as a medicament for promoting weight reduction (e.g., treating obesity) and reduction of fat absorption in mammals because they inhibit lipases in the gastrointestinal tract. As such, they are administered in a manner suitable for reaching the gastrointestinal tract during digestion. They are therefore preferably administered orally as soon as up to about one hour prior to a meal and as late as to up to about one hour subsequent to a meal. Preferably, the polymer is of sufficiently high molecular weight to resist absorption, partially or completely, from the gastrointestinal tract into other parts of the body. The polymers can have molecular weights ranging from about 500 Daltons to about 500,000 Daltons (although the upper bound is not important), preferably from about 2,000 Daltons to about 150,000 Daltons. Often, the molecular weight of crosslinked polymers cannot be determined.

An “effective amount” is the quantity of polymer which results in a greater amount of weight reduction or reduction in fat absorption over a period of time during which a subject is being treated with the polymer drug for obesity compared with the corresponding time period in absence of such treatment. This assumes that a subject's health and diet are similar during the two time periods. Typical dosages range from about 5 milligrams/day to about 10 grams/day, preferably from about 50 milligrams/day to about 5 grams/day. The polymer can be administered alone or in a pharmaceutical composition comprising the polymer and an acceptable carrier or diluent. Typically, the pharmaceutical composition comprises an effective concentration of the polymer, which is a concentration that can administer an effective amount of the polymer.

The precise amount of polymer being administered to a subject will be determined on an individual basis and will depend on, at least in part, the subject's individual characteristics, such as general health, age, sex, body weight and tolerance to drugs, amount of fat consumed and the degree to which the subject is overweight and the amount of weight reduction sought or the amount of reduction in fat absorption sought.

The disclosed polymers can be administered to the subjects in conjunction with an acceptable pharmaceutical carrier or diluent as part of a pharmaceutical composition for treatment of obesity or reducing fat absorption in mammals in need of treatment therefor. Formulations vary according to the route of administration selected (e.g., oral, rectal), but for oral administration are typically capsules or tablets. Solutions, suspensions and emulsions, for example, are also possible.

For oral administration, the polymers disclosed herein can be formulated readily by combining the polymers with pharmaceutically acceptable carriers or diluents well known in the art. Such carriers or diluents enable the polymers of the invention to be formulated as tablets, pills, dragees, capsules, liquids, gels, syrups, slurries, suspensions and the like, for oral ingestion by a patient to be treated. Pharmaceutical preparations for oral use can be obtained by combining the polymer with a solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents can be added, such as the cross-linked polyvinyl pyrrolidone, agar, or alginic acid or a salt thereof such as sodium alginate.

Dragee cores are provided with suitable coatings. For this purpose, concentrated sugar solutions can be used, which can optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments can be added to the tablets or dragee coatings for identification or to characterize different combinations of active compound doses.

Pharmaceutical preparations that can be used orally include push-fit capsules made of a suitable material, such as gelatin, as well as soft, sealed capsules made of a suitable material, for example, gelatin, and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds can be dissolved or suspended in suitable liquids, such as fatty oils, liquid paraffin, or liquid polyethylene glycols. In addition, stabilizers can be added. All formulations for oral administration should be in dosages suitable for such administration. Methods for encapsulating compositions (such as in a coating of hard gelatin or cyclodextran) are known in the art (Baker, et al., “Controlled Release of Biological Active Agents”, John Wiley and Sons, 1986).

Other standard pharmaceutical formulation techniques can be employed, such as those described in Remington's Pharmaceutical Sciences, Mack Publishing Company, Easton, Pa.

An electron withdrawing group is a substituent which results in a phenyl ring that has less electron density when the group is present than when it is absent. Electron withdrawing groups have a Hammett sigma value greater than zero (see, for example, C. Hansch, A. Leo and D. Hoeckman, “Exploring QSAR Hydrophobic, Electronic and Steric Constants”, American Chemical Society (1995), pages 217-32). Examples of electron withdrawing groups represented by X include halogens (e.g., F, Cl, Br, I), —NO₂, —CN and —Y—R, where R is a substituted or unsubstituted straight chained hydrocarbyl group with an ether, thioether, phenylene, amine or ammonium linkage. Examples of electron withdrawing groups represented by Y in structural formulae depicted herein include —CHD-, —CD₂-, —COO—, —CONH—, —CO— and —SO₂—, where D is a halogen.

An electron donating group is a substituent which results in a phenyl ring that has more electron density when the group is present than when it is absent. Electron donating groups have a Hammett sigma value less than zero. Examples of electron donating groups include —NH₂, —NHR, —NR₂, alkyl groups (e.g., —CH₃, —CH₂CH₃), —C₆H₅, —OH, and alkoxy groups (e.g., —OCH₃, —OCH₂CH₃), where R is an alkyl group.

A hydrocarbylene group is an alkylene group, i.e., —(CH₂)ₓ— where x is a positive integer (e.g., between 1 and 30), preferably between 6 and 30 (such as between 8 and 30), more preferably between 6 and 15. Hydrocarbylene groups are optionally interrupted by one or more heteroatoms selected from the group consisting of N, S, and O. “Optionally interrupted” does not include replacing the terminal methylene groups with a heteroatom.

Alkyl groups consist of only carbon and hydrogen, are completely saturated and are monovalent. An alkyl group can be branched or unbranched and cyclic or acyclic. Suitable substituents for an alkyl group are those which do not significantly lower the lipase inhibiting ability of the polymer, for example, do not lower the activity by more than a factor of about two. Examples include aryl, —OH, halogen (—Br, —Cl, —I and —F), —O(R′), —O—CO—(R′), —CN, —NO₂, —COOH, —NH₂, —NH(R′), —N(R′)₂, —COO(R′), —CONH₂, —CONH(R′), —CON(R′)₂, —S(O)R′, —S(O)₂R′, —SH and —S(R′). Each R′ is independently an alkyl group or an aryl group. A substituted alkyl group can have more than one substituent.

Suitable substituents for an alkylene group are identical to those for alkyl groups.

Aryl groups include carbocyclic aromatic groups such as phenyl and naphthyl, heteroaryl groups such as imidazolyl, thienyl, furanyl, pyridyl, pyrimidyl, pyranyl, pyrazolyl, pyrazinyl, thiazolyl, oxazolyl and fused polycyclic aromatic ring systems in which a carbocyclic aromatic ring or heteroaryl ring is fused to one or more other heteroaryl rings (e.g., benzothienyl, benzofuranyl, indolyl, quinolinyl, benzothiazolyl, benzooxazolyl, benzimidazolyl and quinolinyl).

Arylene groups are similar to aryl groups, but are divalent rather than monovalent.

Polymers of the present invention can typically be prepared in three steps, where polymerization typically occurs third. An aryl boronic acid-containing compound is first connected to a compound having a hydrocarbylene group optionally interrupted by one or more heteroatoms to from a precursor that will become the pendant group of the polymer. Syntheses of suitable precursors that are appropriate pendant groups is described in, for example, U.S. Application Nos. 60/302,081 and 10/187,397 (published as US 2003/0064963 A1), the contents of which are incorporated herein by reference. For example, a compound with a carboxylic acid group or an activated carboxylic acid group (e.g., ester, amide, acid chloride) and is reacted with a second compound comprising a nucleophilic group, such as an alcohol or amine group. To prepare a precursor where the aryl boronic acid is linked to a hydrocarbylene group through a thioether, the thioether linkage can be formed by reacting a primary alkyl halide with a primary alkyl thiolate or by reacting a Grignard reagent with a symmetrical disulfide.

The next step in the synthesis involves coupling a polymerizable monomer (e.g., 4-hydroxystyrene, acrylate, acrylamide) to the pendant group of the polymer. A nucleophilic group on the one of the compounds is reacted with a carboxylic acid group or an activated carboxylic group on the second compound. The first two steps of the synthesis can be reversed, such that a polymerizable monomer is coupled to a compound containing a hydrocarbylene group and subsequently attaching a compound containing an aryl boronic acid group. The last step of the synthesis is polymerization of a functionalized monomer with a pendant group, as described above.

Thus, the order of the synthesis is typically: 1) coupling an aryl boronic acid-containing compound to a compound having a hydrocarbylene group interrupted by one or more heteroatoms to form a pendant group, 2) coupling the pendant group to a polymerizable monomer to form a functionalized monomer, and 3) polymerizing the functionalized monomer. Alternatively, the hydrocarbylene linker and aryl boronic group can be added to a pre-assembled polymer backbone using reactions similar to those described above.

One example of this synthesis is represented in Scheme A:

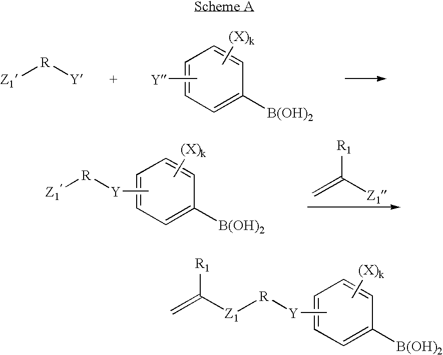

Y′ and Y″ are functional groups which react to form Y. When Y′ is OH or NH₂ and Y″ is COOH or COCl, or the reverse, then Y is an ester or amide, respectively. Alternatively, when Y′ is OH and Y″ is a halogen, or the reverse, then Y is an ether linkage. Specific conditions for carrying out reactions of this type are provided in Examples 8, 9, 10, 12, 16 and 17. Reactions where Y′ or Y″ is COOH require a coupling agent such as those listed in “Advanced Organic Chemistry, Fourth Edition,” by Jerry March and references therein, including a chlorinating agent (e.g., SOCl₂, PCl₃), dicyclohexylcarbodiimide, N,N′-carbonyldiimidazole, POCl₃, TiCl₄, SO₂ClF, benzotriazol-1-yl diethyl phosphate, Ti(O-butyl)₄, N,N,N′,N′-tetramethyl(succinimido)uranium tetrafluoroborate, 1,1′-carbonylbis(3-methylimidazolium) triflate, Lawesson's reagent, chlorosulfonyl isocyanate, and P₂I₄.

Similarly, Z₁′ and Z₁″ are functional groups which react to form Z₁. In many cases, Z₁′ and Z₁″ also involve coupling a hydroxyl or an amine group to a carboxylic acid or forming an ether linkage, as described above. Specific conditions for carrying out reactions of this type are provided in Examples 1-17.

Protecting groups can be used when necessary. Suitable protecting groups are disclosed in “Protective Groups in Organic Synthesis, Third Edition,” by Peter G. M. Wuts and Theodora W. Green, Wiley Interscience: 1999, the contents of which are incorporated by reference.

Copolymers of the present invention can be prepared by a variety of methods known to one of ordinary skills in the art. Random copolymers are prepared by simultaneously polymerizing two or more monomers, such that the product copolymer contains a random distribution of monomeric units, as exemplified in Examples 18-58. Block copolymers are prepared by conjugating two or more different polymeric backbones, for example, a polymer “A” and a polymer “B” can be conjugated to form an “ABABAB” alternating block copolymer or a random block copolymer such as “ABBAAB”. Specific conditions for preparing a block copolymer are provided in Example 60. Graft and comb copolymers are prepared by coupling pendant reactive functional groups on a first polymer with complementary reactive functional groups of a second polymer, such that the second polymer becomes a pendant group on the first polymer. Specific conditions for preparing a graft or comb copolymer are provided in Example 59. Star copolymers are prepared from a central molecule which provides multiple branch points, from which linear polymers emanate (see J.P. Kennedy and B. Ivan, Designed Polymers by Macromolecular Engineering, Hanser Publishers, Munich, Germany, 1991). The linear polymers can be the same or different, and either the whole linear polymer can be attached to the central molecule or the polymer can be synthesized on the central molecule. A dendrimer is comprised of a monomeric unit having a branch point, such that each time the monomer repeats within the dendrimer, a new branch point occurs and a hyperbranched copolymer results (see E. Malmstrom, M. Johansson and A. Hult, Macromolecules, 28, 1698 (1995)).

The following examples are not intended to be limiting in any way.

### EXEMPLIFICATION

### Example 1

### 4-(13′-acryloxy-2′-thia)tridecyl phenyl boronic acid

To a 250-mil, three-necked, round bottomed flask were added 10 g of 4-bromomethylphenyl boronic acid and 100 ml of tetrahydrofuran (THF); After complete dissolution, the solution was degassed by bubbling nitrogen through the reaction mixture for about 30 minutes. While stirring, 9.51 g of 11-mercaptoundecanol was added to this solution under nitrogen. Diisopropylethylamine (23.4 mL) was added via a syringe over 5 minutes. The reaction mixture was kept stirring for 48 hours under nitrogen at room temperature. TLC showed the reaction was complete. The solvent was removed in vacuo, and the residue was partitioned between ethyl acetate (300 mL) and water (150 mL). The organic phase was washed with water (100 mL), 5% hydrochloric acid solution (3×100 mL), water (100 mL), and brine (100 mL). The ethyl acetate solution was dried over sodium sulfate for 30 minutes. After filtration, the solvent was removed in vacuo. The residue was dissolved in minimum amount of ethyl acetate, and the solution was placed in a freezer. The 4-(13′-hydroxy-2′-thia)tridecyl phenyl boronic acid product was crystallized out. After filtration and drying, 13.2 gram of the product was obtained as an off white solid.

To a 500-ml, three-necked, round-bottomed flask were added 14 g of 4-(13′-hydroxy-2′-thia)tridecyl phenyl boronic acid, 16.03 g of diisopropylethylamine, 10 ml of anhydrous dimethylformamide (DMF), and 30 ml of anhydrous dichloromethane. Resulting solution was cooled to 0° C. using an ice bath. Acryloyl chloride (4.48 g) dissolved in 20 ml of anhydrous dichloromethane was added slowly. Under nitrogen atmosphere, the reaction mixture was stirred at 0° C. and was subsequently allowed to warm to room temperature slowly. After stirring at room temperature for 48 hr, dichloromethane was removed under reduced pressure. To the residue was added 300 ml of deionized water and the reaction mixture was extracted with ethyl acetate (3×200 ml). The combined ethyl acetate phase was washed with 5% aqueous HCl solution (3×200 ml), deionized water (2×200 ml) and 200 ml of brine. The organic phase was dried over anhydrous sodium sulfate for 10 minutes and filtered. The volume of the solution was reduced to 200 ml. Upon cooling the concentrated solution at 0° C. the compound crystallized. The residue was 5 filtered and dried under reduced pressure yielding 13 g of the product as an off white solid.

### Example 2

### 4-(14′-Acryloxy-3′-thia-1′-keto)tetradecyl Phenyl boronic Acid

To a 2-liter, three-necked, round bottomed flask fitted with an overhead stirrer were added 100 g of 4-(14′-hydroxy-3′-thia-1′-keto)tetradecyl phenyl boronic acid (the synthesis is described in U.S. Application Nos. 60/302,081 and 10/187,397, published as US 2003/0064963 A1)) and 400 ml of anhydrous tetrahydrofuran (THF) and 57.5 ml of diisopropylethylamine. While stirring the reaction was cooled to 0° C. under nitrogen atmosphere and 29.7 g of acryloyl chloride was added slowly. The reaction mixture was allowed to warm to room temperature slowly and stirring continued for 4 hr at room temperature. Another batch of diisopropylethylamine (11.1 ml) and stirring continued at room temperature. After 24 hr, 11.1 ml of disopropylethylamine and 11.1 ml of acryloyl chloride were added successively to the reaction mixture. Stirring continued for further 24 hr and another batch of acryloyl chloride (7.4 ml) was added. This was followed by addition of 11.1 ml of acryloyl chloride after 6 hr. The reaction mixture was stirred for additional 1 0 hr. At the end of the reaction, the solvent was removed under reduced pressure. To the residue was added 400 ml of deionized water and the resulting suspension was extracted with ethyl acetate (3. x 400 ml). The combined organic phase was washed with 1 N HCl (3×200 ml), deionized water (200 ml), 4% aqueous sodium bicarbonate (2×200 ml), deionized water (200 ml), and brine (200 ml). The organic phased was dried over anhydrous magnesium sulfate for 15 minutes. After filtration, the solvent was removed under reduced pressure. The residue was dissolved in 300 ml of methanol/water mixture (90:10) and was allowed to cool at 0° C. to crystallize the product. The solid crystals were filtered and dried under reduced pressure over phosphorous pentoxide yielding 104 g of an off white solid.

### Example 3

### 4-(14′-methacryloxy-3′-thia-1′-keto)tetradecyl phenyl boronic acid

To a 500 ml, three-necked, round bottomed flask were added 10.29 g of 4-(14′-hydroxy-3′-thia-1′-keto)tetradecyl phenyl boronic acid (the synthesis is described in U.S. Ser. No. 60/302,081), 90 ml of dichloromethane, 20 ml of DMF and 4.9 ml of diisopropylethylamine. The reaction mixture was allowed to cool down to 0° C. using an ice bath. While stirring under nitrogen atmosphere, 4.0 ml of methacryloyl chloride dissolved in 10 ml of dichloromethane was added to the reaction mixture over a period of 25 minutes while maintaining temperature to below 5° C. throughout the course of addition. After 1 hr, additional 4.9 ml of diisopropylethylamine was added. The reaction mixture was allowed to warm up to room temperature slowly and was stirred at room temperature for 16 hr. Methacryloyl chloride (3.0 ml) followed by diisopropylethylamine (3.0 ml) were added and stirring continued for additional 24 hr. The reaction mixture was filtered and the solution was concentrated under reduced pressure. To the resulting oily concentrate was added 150 ml of deionized water and the resulting suspension was extracted with ethyl acetate (3×200 ml). The combined organic phase was washed with 5% aqueous HCl (2×150 ml), 150 ml of deionized water and 150 ml of brine. The organic phase was dried over anhydrous sodium sulfate for 30 minutes. The solvent was removed under pressure. The residue was purified by silica gel column chromatography using dichloromethane/methanol (98:2 v/v) as the mobile phase. After evaporation of the solvent 2.5 g of the product was isolated as off-white solid.

### Example 4

### 4-(12′-acryloxy-3′-thia-1′-keto)dodecyl phenyl boronic acid

To a 250 ml, three-necked, round bottomed flask were added 10 g of 4-(12′-hydroxy-3′-thia-1′-keto)dodecyl phenyl boronic acid (the synthesis is described in U.S. Ser. No. 60/302,081), 50 ml of THF, and 5 ml of diisopropylethylamine. The reaction mixture was allowed to cool down to 0° C. using an ice bath. While stirring under nitrogen atmosphere, 2.6 ml of acryloyl chloride dissolved in 5 ml of THF was added to the reaction mixture over a period of 25 minutes. The temperature of the reaction mixture was maintained at ˜5° C. throughout the course of addition. The reaction mixture was allowed to warm up to room temperature slowly and was stirred at room temperature for 24 hr. Acryloyl chloride (0.5 ml) followed by diisopropylethylamine (0.5 ml) were added and stirring continued for additional 48 hr. The reaction mixture was filtered and the solution was concentrated under reduced pressure. To the resulting oily concentrate was added 150 ml of deionized water and the resulting suspension was extracted with ethyl acetate (3×200 ml). The combined organic phase was washed with 5% aqueous HCl (2×150 ml), 150 ml of deionized water and 150 ml of brine. The organic phase was dried over anhydrous sodium sulfate for 30 minutes. The solvent was removed under pressure. The residue was purified by column chromatography on silica using dichloromethane/methanol (98:2 v/v) as the mobile phase. Removal of the solvent under reduced pressure yielded 5 g of the product as viscous oil.

### Example 5

### 4-(9′-Acryloxy-3′-thia-1′-keto)nonyl phenyl boronic Acid

To a 250 ml, three-necked, round bottomed flask were added 8 g of 4-(9′-hydroxy-3′-thia-1′-keto)nonyl phenyl boronic acid (the synthesis is described in U.S. Ser. No. 60/302,081), 40 ml of THF, and 7 ml of diisopropylethylamine. The reaction mixture was allowed to cool down to 0° C. using an ice bath. While stirring under nitrogen atmosphere, 3.29 ml of acryloyl chloride in 5 ml of THF was added to the reaction mixture over a period of 25 minutes. The temperature of the reaction mixture was not allowed to exceed 5° C. throughout the course of addition. The reaction was allowed to warm up to room temperature slowly and was stirred at room temperature for 24 hr. Acryloyl chloride (1.5 ml) followed by diisopropylethylamine (3.5 ml) were added and stirring continued for additional 48 hr. The reaction mixture was filtered and the solution was concentrated under reduced pressure. To the resulting oily concentrate was added 150 ml of deionized water and the resulting suspension was extracted with ethyl acetate (3×200 ml). The combined organic phase was washed with 5% aqueous HCl (2×150 ml), 150 ml of deionized water, and 150 ml of brine. The organic phase was dried over anhydrous sodium sulfate for 30 minutes. The solvent was removed under pressure. The residue was purified by column chromatography on silica using dichloromethane/methanol (98:2 v/v) as the mobile phase. Removal of the solvent under reduced pressure yielded 3.8 g of the product as viscous oil.

### Example 6

### 4-(14′-acryloxy-3′-thia-1′-keto)tetradecyl-3-Fluoro phenyl boronic acid

To a 250 ml, three-necked, round bottomed flask were added 3.4 g of 4-(14′-hydroxy-3′-thia-1′-keto)tetradecyl-3-fluoro phenyl boronic acid (the synthesis is described in U.S. Ser. No. 60/302,081), 20 ml of THF, and 1.53 ml of diisopropylethylamine. The reaction mixture was allowed to cool down to 0° C. using an ice bath. While stirring under nitrogen atmosphere, 0.71 ml of acryloyl chloride in 5 ml of THF was added to the reaction mixture over a period of 5 minutes. The temperature of the reaction mixture was not allowed to exceed 5° C. throughout the course of addition. The reaction was allowed to warm up to room temperature slowly and was stirred at room temperature for 24 hr. Diisopropylethylamine (0.3 ml) and acryloyl chloride (0.14 ml) were added slowly and stirring continued for 24 hr. Finally, 0.5 ml of diisopropylethylamine and 0.2 ml of acryloyl chloride were added and the reaction mixture was allowed to stir for additional 48 hr. The reaction mixture was filtered and the solution was concentrated under reduced pressure. To the resulting. oily concentrate was added 150 ml of deionized water and the resulting suspension was extracted with ethyl acetate (3×200 ml). The combined organic organic phase was washed with 5% aqueous HCl (2×150 ml), 150 ml of deionized. water and 150 ml of brine. The organic phase was dried over anhydrous sodium sulfate for 30 minutes. The solvent was removed under pressure. The residue was purified by column chromatography on silica using dichloromethane/methanol (98:2 v/v) as the mobile phase. Removal of the solvent under reduced pressure yielded 2.8 5 g of the product as viscous oil.

### Example 7

### 4-(1 4′-Styroxy-3′-thia-1′-keto)tetradecyl phenylboronic Acid

To a 250 ml, three- necked, round-bottomed flask were added 0.91 g of 4-hydroxy styrene, 3 g of 4-(14′-bromo-3′-thia-1′-keto)tetradecyl phenyl boronic acid (the synthesis is described in U.S. Ser. No. 60/302,081), 1.45 g of potassium carbonate, 100 mg of sodium iodide and 50 ml of anhydrous acetone. The reaction mixture heated to reflux under a nitrogen atmosphere for 30 hr. After cooling down to room temperature, it was filtered and the residue was washed with 20 ml of acetone. The combined filtrate was concentrated under reduced pressure. The residue was dissolved in 30 ml of ethyl acetate and the resulting solution was washed with 5% aqueous sodium bicarbonate solution (2×250 ml), deionized water (2×250 ml), and 100 ml brine. The organic phase was dried over anhydrous sodium sulfate for 30 minutes. After filtration, the solution was evaporated to dryness yielding 2 g of viscous oil.

### Example 8

### Synthesis of 6-(4′-vinyl)phenoxy hexyl (4″-boronato)benzoate

Synthesis of this compound involves the following three steps.

8a. Synthesis of 6-(4′-vinyl)phenoxy hexanol. To a 250 ml, three necked, round-bottomed flask were added 3g of 4-hydroxy styrene, 3.62 g of 6-bromohexanol, 5.5 g of potassium carbonate, 100 mg of sodium iodide, and 70 ml of anhydrous acetone. The reaction mixture was heated to reflux for 48 hr under nitrogen atmosphere. After cooling down to room temperature, it was filtered. The solvent was removed under reduced pressure. The residue was dissolved in 50 ml of diethylether and the solution was washed with 5% aqueous sodium hydroxide (2×200 ml), deionized water (2×200 ml) and 100 ml of brine. After drying over magnesium sulfate for 30 minutes the solvent was removed under reduced pressure. The residue was recrystallized from ethanol yielding 3 g of an off-white solid.

8b. Synthesis of 6-(4′-vinyl)henoxy hexyl (4″-iodo)benzoate. To a 250 ml, three necked, round-bottomed flask were added 2.4 g of 6-(4′-vinyl)phenoxy hexanol, 3 ml of triethylamine, 100 mg of 4(N,N-dimethyl)amino pyridine, and 30 ml of dichloromethane. The resulting solution was cooled to below 5° C. using an ice bath. To this solution was added 3.2 g of 4-iodo benzoyl chloride dissolved in 10 ml of dichloromethane over a period of 15 minutes. The reaction mixture was slowly allowed to warm up to room temperature and was stirred for 48 hr. The reaction mixture was extracted with deionized water (2×100 ml), 0.5 N HCl (2×100 ml), deionized water (100 ml), 5% aqueous sodium bicarbonate (2×100 ml), deionized water (100 ml), and brine (100 ml). After drying over anhydrous sodium sulfate for 30 minutes, the solvent was removed under reduced pressure. The residue was chromatographed on silica gel using ethyl acetate:hexane (1:4 v/v) as the mobile phase. Removal of the solvent under reduced pressure offered 2.6 g of the product as viscous oil.

8c. 6-(4′-vinyl)phenoxy hexyl (4″-boronato)benzoate. To an oven-dried, 250 ml, three necked, round-bottomed flask were added 2.5 g of 6-(4′-vinyl)phenoxy hexyl (4″-iodo)-benzoate and 50 ml of anhydrous THF. While stirring under nitrogen atmosphere, the reaction mixture was cooled to −70° C. While maintaining the temperature at −70° C., 2.9 ml of 2M solution (in THF) of isopropyl magnesium bromide was added slowly to the reaction mixture. After stirring at this temperature for 2 hr, 577 mg of trimethyl borate in 3 ml of THE was added to the reaction mixture. The stirring continued for 16 hr, during which time the temperature of the reaction was slowly allowed to warm up to room temperature. To the reaction mixture was added 25 ml of 0.5 N HCl and stirred for 30 minutes. The reaction mixture was extracted with diethyl ether (2×100 ml). The combined organic phase was washed with deionized water (2×200 ml) and 50 ml of brine. After drying over anhydrous sodium sulfate for 30 minutes the solvent was removed under reduced pressure. The residue was recrystallized from ethyl acetate:hexane (7:3) yielding 1.25 g of the product as an off white solid.

### Example 9

### Synthesis of 12-(4′-vinyl)phenoxy dodecyl(4″-boronato)benzoate

Synthesis of this compound involves the following three steps.

9a. Synthesis of 12-(4′-vinyl)phenoxy dodecanol. To a 250 ml, three-necked, round-bottomed flask were added 4.5 g of 4-hydroxystyrene, 5 g of 12-bromododecanol, 7 g of potassium carbonate, 100 mg of sodium iodide, and 70 ml of anhydrous acetone. The reaction mixture was heated to reflux for 48 hr under nitrogen atmosphere. After cooling down to room temperature, it was filtered. The solvent was removed under reduced pressure. The residue was dissolved in 250 ml of ethyl acetate and the solution was washed with 5% aqueous sodium hydroxide (2×200 ml), deionized water (2×200 ml) and 100 ml of brine. After drying over magnesium sulfate for 30 minutes the solvent was removed under reduced pressure. The residue was recrystallized from ethanol yielding 4.2 g of an off-white solid.

9b. Synthesis of 1 2-(4′-vinyl)phenoxy dodecyl (4″-iodo)benzoate. To a 250 ml, three necked, round-bottomed flask were added 2.0 g of 12-(4′-vinyl)phenoxy dodecanol, 3.5 ml of diisopropylethylamine, 25 mg of 4(N,N-dimethyl)amino pyridine, and 20 ml of THF. The resulting solution was cooled to below 5° C. using an ice bath. To this solution was added 2.0 g of 4-iodobenzoyl chloride dissolved in 10 ml of THF over a period of 15 minutes. The reaction mixture was slowly allowed to warm up to room temperature and was stirred for 48 hr. After adding 30 mL of ethyl acetate, the reaction mixture was extracted with deionized water (2×100 ml), 0.5 N HCl (2×100 ml), deionized water (100 ml), 5% aqueous sodium bicarbonate (2×100 ml), deionized water (100 ml), and brine (100 ml). After drying over anhydrous sodium sulfate for 30 minutes, the solvent was removed under reduced pressure. The residue was recrystallized from ethanol yielding 2.2 g of the product as an off white solid.

9c. Synthesis of 12-(4′-vinyl)phenoxy dodecyl (4″-boronato)benzoate. To an oven-dried, 250 ml, three-necked, round-bottomed flask were added 2.0 g of 12-(4′-vinyl)phenoxy dodecyl-(4″-iodo)-benzoate and 40 ml of anhydrous THF. While stirring under nitrogen atmosphere, the reaction mixture was cooled to −70° C. While maintaining the temperature at −70° C., 2.7 ml of 2M solution (in THF) of isopropyl magnesium bromide was added slowly to the reaction mixture. After stirring at this temperature for 2 hr, 570 mg of trimethyl borate dissolved in 3 ml of THF was added to the reaction mixture. The stirring continued for 16 hr, during which time the reaction was slowly allowed to warm up to room temperature. To the reaction mixture was added 25 ml of 0.5 N HCl and stirred for 30 minutes. The reaction mixture was extracted with diethyl ether (2×100 ml). The organic phase was washed with deionized water (2×200 ml) and 50 ml of brine. After drying over anhydrous sodium sulfate for 30 minutes the solvent was removed under reduced pressure. The residue was recrystallized from ethanol:water (9:1) yielding 1.1 g of the product as an off white solid

### Example 10

### Synthesis of N-(3-boronato)phenyl 10-undecenamide

To a 500 ml, three necked, round-bottomed flask were added 12 g of 3-amino phenyl boronic acid hemisulfate and 80 ml of dichloromethane. The reaction mixture was cooled to 0° C. and 22.6 ml of diisopropylethylamine was added slowly to it. While stirring at 0° C., 16.4 ml of 1 0-undecenoyl chloride in 20 ml of dichloromethane was added dropwise to the reaction mixture over three minutes. After complete addition of the acid chloride, the temperature was slowly allowed rise up to room temperature and the reaction mixture was stirred at room temperature for 18 hr. The solvent was removed under reduced pressure and the residue was treated with 150 ml of deionized water. The resulting suspension was extracted with ethyl acetate (3×150 ml) and the combined organic phase was washed with 5% aqueous sodium bicarbonate (2×150 ml), 200 ml of deionzied water and 100 ml of brine. The organic phase was dried over sodium sulfate for 15 minutes. The product was crystallized by concentrating the solution volume to 100 ml, and keeping the resulting solution in the refrigerator. Filtration and drying of the residue offered 10 g of the product as an off white solid.

### Example 11

### Synthesis of N-(3′-boronato)phenyl (14-acrylamido-12-thia)tetradecylamide

The synthesis of this compound is accomplished through the following two steps.

11a. Synthesis of N-(3′-boronato)phenyl (14-amino-12-thia) tetradecylamide. To a 250 ml, three-necked, round-bottomed flask were added 2 g of N-(3-boronato)phenyl 10-undecenamide (example 10), 0.93 g of cystamine hydrochloride, 50 mg of AIBN and 30 ml of methanol. The reaction mixture was heated to reflux for 10 hr under nitrogen atmosphere. Another batch of cystamine hydrochloride (1.0 g) and AIBN (20 mg) were added and the refluxing continued for additional 14 hr. After cooling to room temperature, the residue was recrystallized from ethyl acetate yielding 2.2 g of the compound as a white powder.

11b. Synthesis of N-(3′-boronato)phenyl (14-acrylamido-12-thia)tetradecylamide. To a 250 ml, three-necked, round-bottomed flask were added 1.5 g of N-(3-boronato)phenyl (14-amino-12-thia)tetradecylamide, 20 ml of dichloromethane, 1 ml of DMF, and 1.32 ml of trimethylamine. The solution was stirred at 0° C. and 0.3 ml of acryloyl chloride dissolved in 2 ml of dichloromethane was added to it. The reaction mixture was allowed to warm up to room temperature slowly and was stirred at this temperature for 14 hr. The reaction mixture was washed with 5% aqueous sodium bicarbonate (2×150 ml), 100 ml of deionized water, 100 ml of 0.5 N HCl, 100 ml of deionized water, and 100 ml of brine. The organic phase was dried over sodium sulfate for 15 minutes. After filtration, the solvent was removed under reduced pressure. The residue was recrystallized from ethyl acetate/hexane yielding 1.2 of the compound as a white crystalline solid.

### Example 12

### Synthesis of N-(3′-boronato)phenyl (1-acrylamido)undecylamide

The compound was synthesized in two steps.

12a. Synthesis of N-(3′-boronato)phenyl 11- aminoundecylamide hydrochloride. To a 250 ml, three necked, round bottomed flask were added 6.75 g of 11-(N-tert-butoxycarbonyl)-aminoundecanoic acid, 5.55 g of 1,3-dicyclohexylcabodiimide, 3.65 g of 1-hydroxybenzotriazole, and 50 ml of DMF. The resulting solution was stirred at 0° C. for 2 hr. At this time, 5 g of 3-aminophenylboronic acid hemisulfate and 3.5 g of diisopropylethylamine were added to the above solution and the resulting reaction mixture was stirred first at 0° C. for 1 hr and subsequently at room temperature for 48 hr. After filtering off the solid, deionized water was added to the solution to precipitate a solid. After drying this residue it was recrystallized from hot ethyl acetate yielding 6.4 g of a white solid.

To 5 g of the above solid dissolved in 20 ml of dioxane was added 9 ml of 4N HCl in dioxane. After stirring for 2 hr at room temperature, 15 ml of ethanol and another 9 ml of 4N HCl were added to the reaction mixture. The resulting reaction mixture was stirred for additional 2 hr. To this reaction mixture was added 200 ml of diethyl ether and the solution was kept at 0° C. to crystallize the product. Filtration and drying of the solid offered 4.2 g of the product as a white solid.

12b. Synthesis of N-(3′-boronato)phenyl (11-acrylamido)undecylamide. To a 100 ml, three-necked, round-bottomed flask were added 3 g of N-(3′-boronato)phenyl 11-aminoundecylamide hydrochloride, 4.4 ml of diisopropylethylamine, 15 ml of dichloromethane and 3 ml of DMF. The reaction mixture was cooled to 0° C. While stirring, 0.82 ml of acryloyl chloride in 3 ml of dichloromethane was added slowly to the reaction mixture. The resulting reaction mixture was allowed to warm up to room temperature slowly and was stirred for 14 hr. The reaction mixture was washed with 5% aqueous sodium bicarbonate (2×150 ml), 100 ml of deionized water, 100 ml of 0.5 N HCl, 100 ml of deionized water, and 100 ml of brine. The organic phase was dried over sodium sulfate for 15 minutes. After filtration, the solvent was removed under reduced pressure. The residue was recrystallized from ethyl acetate/hexane yielding 2.8 g of the product as a white solid.

### Example 13

### Synthesis of 2-(4′-Vinyl) Phenoxy Acetylphenyl Boronic Acid Neopentyl Glycol Ester

To a 50 ml, round-bottomed flask were added 0.58 g of 4-hydroxy styrene, 1.5 g of 4-bromoacetyl phenyl boronic acid neopentyl glycol ester, 0.73 g of potassium carbonate, and 15 ml of anhydrous acetone. The reaction mixture was stirred at refluxing temperature under nitrogen atmosphere for 6 hr. After cooling down to room temperature, the reaction mixture was filtered. The solvent was removed under reduced pressure. The residue was dissolved in 15 ml of hexane and 1 ml of dichloromethane. Cooling the solution in the refrigerator resulted in crystallization of the product. Filtration and drying of the residue yielded 0.5 g of the product as an off white solid.

### Example 14

### Synthesis of Neopentyl Glycol Protected (4′-Boronato)acetylphenyl (4-Vinyl) benzoate

To a 50 ml, round-bottomed flask were added 2.1 g of 4-bromoacetyl phenyl. boronic acid neopetyl glycol ester, 1 g of 4-vinyl benzoic acid, 0.9 g of potassium carbonate and 10 ml of anhydrous DMF. The reaction mixture was stirred at room temperature for 2 hr. The reaction mixture was filtered and the residue was washed with 15 ml of dichloromethane. The combined organic phase evaporated to dryness under reduced pressure. The residue was dissolved in 10 ml of THF and to this solution was added 15 ml of 10% aqueous HCl. After stirring for 24 hr at room temperature, THF was removed under reduced pressure. Cooling the aqueous solution in the refrigerator led to crystallization of the product. Filtration and drying yielded 0.57 g of the product a white crystalline solid.

### Example 15

### Synthesis of Neopentyl Glycol Protected (4′-Boronato)acetylphenyl Acrylate

To a 50 ml, round-bottomed flask were added 2.05 g of 4-bromoacetyl phenyl boronic acid neopentyl glycol ester, 0.45 ml of acrylic acid, 0.91 g of potassium carbonate and 10 ml of anhydrous DMF. The reaction mixture was stirred at room temperature for 1.5 hr. The reaction mixture was filtered and the residue was washed with 15 ml of diethyl ether. The combined organic phase was washed with brine. The organic phase evaporated to dryness under reduced pressure. The product was purified by column chromatography on silica gel using ethyl acetate:hexane (1:4, v/v) as the mobile phase yielding 1.0 g of the product as a viscous oil.

### Example 16

### Synthesis of 11-acryloxy undecyl (4′-boronato) benzoate

This compound was synthesized in two steps.

16a. Synthesis of 11-bromoundecyl acrylate. To a 500 ml, three-necked, round-bottomed flask were added 5.66 g of acryloyl chloride and 60 ml of anhydrous THF. The solution was cooled using an ice bath. A solution of 12.8 g of 11-bromo-1-undecanol and 6.06 g of triethylamine in 80 ml of anhydrous THE was added slowly to the cold acryloyl chloride solution. After completion of addition, the reaction mixture was slowly warmed up to room temperature and was stirred at room temperature for 16 hr. The reaction mixture was filtered and the residue was washed with 20 ml of THF. The combined filtrate evaporated to dryness under reduced pressure. The residue was purified by column chromatography using hexane:ethyl acetate (7:3, v/v). Removal of the solvent under reduced pressure yielded 6.0 g of the product as viscous oil.

16b. Synthesis of 11-acryloxy undecyl (4′-boronato) benzoate. In a 100 ml, round bottomed flask were taken 5 g of 11-bromoundecyl acrylate, 2.7 g of 4-carboxybenzene boronic acid, 7 g of potassium hydrogen carbonate, 100 mg of 3,5-di-tert-butyl-4-hydroxyanisole, and 50 ml of anhydrous DMF. The reaction mixture was stirred at 60° C. for 22 hr under nitrogen atmosphere. After cooling down to room temperature, the reaction mixture was filtered. The solvent was removed under reduced pressure. The residue was dissolved in 200 ml of ethyl acetate and solution was washed with 10% sodium bicarbonate solution (3×100 ml), deionized water (2×100 ml) and 100 ml of brine. The organic phase was dried over anhydrous sodium sulfate for 30 minutes. After filtration, the solvent was removed under reduced pressure. The residue was purified by column chromatography using hexane/ethyl acetate (6:4, v/v) as the mobile phase. Removal of the solvent under reduced pressure offered the 3.8 g of the product as an off white solid.

### Example 17

### Synthesis of 11-acryloxy-3,6,9-trioxa undecyl (4′-boronato)benzoate

Synthesis of this compound involves three synthetic steps.

17a. Synthesis of tetra(ethylene glycol) monotosylate. To a 3 L, three necked, round-bottomed flask were added 388.4 g of tetra(ethylene glycol), 95.2 g of p-toluene sulfonyl chloride, and 1 L of dichloromethane. After cooling the solution to 0° C. using an ice bath, 139.3 ml of triethylamine and 2.44 g of 4-(N,N-dimethyl)aminopyridine was added. The resulting reaction mixture was stirred under nitrogen atmosphere at room temperature for 16 hr. The reaction mixture was filtered off and the filtrate was washed with deionized water (2×300 ml), 2N HCl (2×300 ml), saturated sodium hydrogencarbonate (2×300 ml) and brine (2×300 ml). The washed organic phase was dried over magnesium sulfate for 1 hr. The magnesium sulfate was removed by filtration and the solvent was removed from the filtrate under reduced pressure yielding 128.4 of the product as a viscous oil.

17b. Synthesis of 11-acryloxy 3,6,9-trioxa undecyl tosylate. To a 250 ml, three necked, round-bottomed flask were added 5.0 g of tetra(ethylene glycol) monotosylate, 2.32 ml of pyridine and 50 ml of THE. After cooling the reaction mixture to 0° C. using an ice bath, 2.33 ml of acryloyl chloride was added slowly. The resulting reaction mixture was stirred at room temperature for 16 hr. After diluting with 100 ml of diethyl ether, the reaction mixture was washed with 0.1 HCl (2×500 ml), saturated sodium hydrogen carbonate (2×500 ml) and 500 ml of brine. The organic phase was collected and was dried over magnesium sulfate for 1 hr. After removal of magnesium sulfate by filtration, the solvent was removed under reduced pressure. The residue was chromatographed on silica gel using hexane/ethyl acetate (1:1, v/v) as the mobile phase. Upon evaporation of the solvent 2.4 g of the product was obtained as a viscous oil.

17c. Synthesis of 11-acryloxy-3,6,9-trioxa undecyl (4′-boronato)benzoate. To a 250 ml, three necked, round bottomed flask were added 2.0 g of 11-acryloxy 3,6,9-trioxaundecyl tosylate, 1.0 g of 4-carboxyphenyl boronic acid, 1.7 g of potassium hydrogen carbonate, 20 mg of 3,5-di-tert-butyl-4-hydroxyanisole, and 100 ml of anhydrous DMF. The reaction mixture was stirred at 60° C. for 15 hr under nitrogen atmosphere. After cooling down to room temperature the reaction mixture was filtered to remove solid residues. The solvent was removed under reduced pressure and the residue was dissolved in 500 ml of ethyl acetate. The resulting solution was extracted with saturated sodium hydrogen carbonate (2×500 ml) and 500 ml of brine. The organic phase was dried over magnesium sulfate for 1 hr. After removal of magnesium sulfate by filtration, the solvent was removed under reduced pressure. The compound was purified by column chromatography on silica gel using ethyl acetate/hexane (7:3, v/v) as the mobile phase. Removal of the solvent yielded 1.2 g of the product as a viscous oil.

### Example 18

### Synthesis of Poly{4-(13′-acryloxy-2′-thia)tridecyl phenyl boronic acid}

To a 25 ml, round bottomed flask were added 1.0 g of 4-(13′-acryloxy-2′-thia)tridecyl phenyl boronic acid, 20 mg of AIBN, and 5 ml of ethanol. The reaction mixture was bubbled with a slow stream of nitrogen for 30 minutes. While stirring the reaction mixture was heated to 65° C. and was stirred at this temperature for 24 hr under nitrogen atmosphere. After the addition of 10 mg of AIBN, beating continued for additional 24 hr. After cooling down to room temperature, the reaction mixture was treated with 30 ml of diethyl ether and was stirred for 20 minutes. Removal of the solvent by filtration and drying of the residue under vacuum offered 300 mg of the polymer as an off white solid.

### Example 19

### Synthesis of Poly{4-(13′-acryloxy-2′-thia)tridecyl phenyl boronic acid-co- sodium 2-acrylamido-2-methyl-1-propanesulfonate}

To a 25 ml, round bottomed flask were added 500 mg of 4-(13′-acryloxy-2′-thia)tridecyl phenyl boronic acid, 62 mg of sodium 2-acrylamido-2-methyl-1-propanesulfonate, 15 mg of AIBN, and 5 ml of ethanol. The reaction mixture was bubbled with a slow stream of nitrogen for 30 minutes. While stirring the reaction mixture was heated to 65° C. and was stirred at this temperature under nitrogen atmosphere. Aliquots of AIBN (5 mg each) were added after an interval of 24 and 48 hrs. The heating continued for a total period of 72 hr. After cooling down to room temperature, the reaction mixture was treated with 30 ml of diethyl ether and was stirred for 20 minutes. The solvent was removed by filtration. The residue was redissolved in 5 ml of methanol and was precipitated from 30 ml of diethyl ether. The solvent was removed by filtration and the residue was dried under vacuum yielding 155 mg of the polymer as an off white solid.

### Example 20

### Synthesis of Poly{4-(13′-acryloxy-2′-thia)tridecyl phenyl boronic acid-co-N-isopropyl acrylamide}

To a 25 ml, round bottomed flask were added 1 g of 4-(13′-acryloxy-2′-thia)tridecyl phenyl boronic acid, 860 mg of N-isopropyl acrylamide, 20 mg of AIBN, and 20 ml of anhydrous 1,4-dioxane. The reaction mixture was bubbled with a slow stream of nitrogen for 30 minutes. While stirring the reaction mixture was heated to 65° C. and was stirred at this temperature under nitrogen atmosphere. The heating continued for 48 hr. After cooling down to room temperature, the reaction mixture was treated with 100 ml of diethyl ether and was stirred for 20 minutes. The solvent was removed by filtration. The residue was redissolved in 10 ml of 1,4-dioxane and was precipitated from 100 ml of diethyl ether. The solvent was removed by filtration and the residue was dried under vacuum yielding 1.5 g of the polymer as an off white solid.

### Example 21

### Synthesis of Poly{4-(13′-acryloxy-2′-thia)tridecyl phenyl boronic acid-co- N,N-diethyl acrylamide}

To a 25 ml, round bottomed flask were added 310 mg of 4-(13′-acryloxy-2′-thia)tridecyl phenyl boronic acid, 300 mg of N,N-diethyl acrylamide, 10 mg of AIBN, and 4 ml of anhydrous 1,4-dioxane. The reaction mixture was bubbled with a slow stream of nitrogen for 30 minutes. While stirring the reaction mixture was heated to 65° C. and was stirred at this temperature under nitrogen atmosphere. The heating continued for 24 hr. After cooling down to room temperature, the reaction mixture was treated with 50 ml of diethyl ether and was stirred for 20 minutes. The solvent was removed by filtration. The residue was redissolved in 10 ml of 1,4-dioxane and was precipitated from 100 ml of diethyl ether. The solvent was removed by filtration and the residue was dried under vacuum yielding 450 mg of the polymer as an off white solid.

### Example 22

### Synthesis of Poly{4-(13′-acryloxy-2′-thia)tridecyl phenyl boronic acid-co- N,N-diethyl acrylamide}

To a 25 ml, round bottomed flask were added 310 mg of 4-(13′-acryloxy-2′-thia)tridecyl phenyl boronic acid, 100 mg of N,N-diethyl acrylamide, 8 mg of AIBN, and 4 ml of isopropyl alcohol. The reaction mixture was bubbled with a slow stream of nitrogen for 30 minutes. While stirring the reaction mixture was heated to 65° C. and was stirred at this temperature under nitrogen atmosphere. The heating continued for 24 hr. After cooling down to room temperature, the reaction mixture was treated with 50 ml of diethyl ether and was stirred for 20 minutes. The solvent was removed by filtration. The residue was redissolved in 10 ml of 1,4-dioxane and was precipitated from 100 ml of diethyl ether. The solvent was removed by filtration and residue was dried under vacuum yielding 300 mg of the polymer as an off white solid.

### Example 23

### Synthesis of Poly{4-(13′-acryloxy-2′-thia)tridecyl phenyl boronic acid-co- N,N-diethyl acrylamide-co-(3-acrylamidopropyl)trimethylammonium chloride}

To a 25 ml, round bottomed flask were added 500 mg of 4-(13′-acryloxy-2′-thia)tridecyl phenyl boronic acid, 97 mg of N-diethyl acrylamide, 105 mg of (3-acrylamidopropyl)-trimethylammonium chloride, 14 mg of AIBN, and 4 ml of ethanol. The reaction mixture was bubbled with a slow stream of nitrogen for 30 minutes. While stirring the reaction mixture was heated to 65° C. and was stirred at this temperature under nitrogen atmosphere. The heating continued for 48 hr. After cooling down to room temperature, the reaction mixture was treated with 50 ml of diethyl ether and was stirred for 20 minutes. The solvent was removed by filtration. The residue was redissolved in 10 ml of ethanol and was precipitated from 100 ml of diethyl ether. The solvent was removed by filtration and residue was dried under 5 vacuum yielding 330 mg of the polymer as an off white solid.

### Example 24

### Synthesis of Poly{4-(13′-acryloxy-2′-thia)tridecyl phenyl boronic acid-co-acrylamide-co-(3-acrylamidopropyl)trimethylammonium chloride}

To a 25 ml, round bottomed flask were added 300 mg of 4-(13′-acryloxy-2′-thia)tridecyl phenyl boronic acid, 380 mg of acrylamide, 320 mg of (3-acrylamidopropyl)trimethylammonium chloride, 10 mg of AIBN, and 5 ml of ethanol. The reaction mixture was bubbled with a slow stream of nitrogen for 30 minutes. While stirring the reaction mixture was heated to 65° C. and was stirred at this temperature under nitrogen atmosphere. After 1 hr of heating, 5 ml of ethanol and 10 mg of AIBN were added and the heating continued for 48 hr. After cooling down to room temperature, the reaction mixture was treated with 50 ml of diethyl ether and was stirred for 20 minutes. The solvent was removed by filtration. The residue was redissolved in 10 ml of ethanol and was precipitated from 100 ml of diethyl ether. The solvent was removed by filtration and residue was dried under vacuum yielding 900 mg of the polymer as an off white solid.

### Example 25

### Synthesis of Poly{4-(14′-acryloxy-3′-thia-1′-keto)tetradecyl phenyl boronic acid}

To a 25 ml, round-bottomed flask were added 1.6 g of 4-(14′-acryloxy-3′-thia-1′-keto)tetradecyl phenyl boronic acid, 10 mg of AIBN, 0.5 ml of 1,4-dioxane, and 5 ml of ethanol. The reaction mixture was bubbled with a slow stream of nitrogen for 30 minutes. While stirring the reaction mixture was heated to 65° C. and was stirred at this temperature under nitrogen atmosphere. The heating continued for 48 hr. After cooling down to room temperature, the reaction mixture was treated with 50 ml of diethyl ether and was stirred for 20 minutes. The solvent was removed by filtration. The residue was redissolved in 10 ml of THF and was precipitated from 100 ml of diethyl ether. The solvent was removed by filtration and residue was dried under vacuum yielding 630 mg of the polymer as an off white solid.

### Example 26

### Synthesis of Poly{4-(14′-acryloxy-3′-thia-1′-keto)tetradecyl phenyl boronic acid-co-acrylamide}

To a 25 ml, round bottomed flask were added 500 mg of 4-(14′-acryloxy-3′-thia-1′-keto)tetradecyl phenyl boronic acid, 22 mg of acrylamide, 5 mg of AIBN, and 5 ml of ethanol. The reaction mixture was bubbled with a slow stream of nitrogen for 30 minutes. While stirring the reaction mixture was heated to 65° C. and was. stirred at this temperature under nitrogen atmosphere. The heating continued for 48 hr. Another batch of AIBN (5 mg) was added and the heating continued for additional 24 hr. After cooling down to room temperature, the reaction mixture was treated with 50 ml of diethyl ether and was stirred for 20 minutes. The solvent was removed by filtration. The residue was redissolved in 10 ml of ethanol and was precipitated from 100 ml of diethyl ether. The solvent was removed by filtration and residue was dried under vacuum yielding 275 mg of the polymer as an off white solid.

### Example 27

### Synthesis of Poly{4-(14′-acryloxy-3′-thia-1′-keto)tetradecyl phenyl boronic acid-co-(3-acrylamidopropyl)trimethylammonium chloride}

To a 25 ml, round bottomed flask were added 420 mg of 4-(14′-acryloxy-3′-thia-1′-keto)tetradecyl phenyl boronic acid, 206 mg of (3-acrylamidopropyl)-trimethylammonium chloride, 5 mg of AIBN, and 5 ml of ethanol. The reaction mixture was bubbled with a slow stream of nitrogen for 30 minutes. While stirring the reaction mixture was heated to 65° C. and was stirred at this temperature under nitrogen atmosphere. The heating continued for 48 hr. Another batch of AIBN (5 mg) was added and the heating continued for additional 24 hr. After cooling down to room temperature, the reaction mixture was treated with 50 ml of diethyl ether and was stirred for 20 minutes. The solvent was removed by filtration. The residue was redissolved in 10 ml of ethanol and was precipitated from 100 ml of diethyl ether. The solvent was removed by filtration and residue was dried under vacuum yielding 240 mg of the polymer as an off white solid.

### Example 28

### Synthesis of Poly{4-(14′-acryloxy-3′-thia-1′-keto)tetradecyl phenyl boronic acid-co-sodium 2-acrylamido-2-methyl-1-propanesulfonate}

Copolymers of this type were prepared in varying compositions of both the monomers. A general procedure for the synthesis of one of the copolymers is described here. Compositions of polymerization mixtures and yields of these copolymers are given in Table 1.

To a 50 ml, three necked, round bottomed flask were added 2.2 g of 4-(14′-acryloxy-3′-thia-1′-keto)tetradecyl phenyl boronic acid, 290 mg of sodium 2-acrylamido-2-methyl-1-propanesulfonate, 14 mg of AIBN, and 10 ml of ethanol. The reaction mixture was bubbled with a slow stream of nitrogen for 30 minutes. While stirring the reaction mixture was heated to 65° C. and was stirred at this temperature under nitrogen atmosphere for 24 hr. Additional batches of AIBN (11 mg each) were added after 24 hr and 48 hr. The heating continued for a total period of 68 hr. After cooling down to room temperature, the reaction mixture was treated with 100 ml of diethyl ether and was stirred for 20 minutes. The solvent was removed by filtration. The residue was redissolved in 10 ml of THF and was precipitated from 100 ml of diethyl ether. The solvent was removed by filtration and residue was dried under vacuum yielding 1.25 g of the polymer as an off white solid.

TABLE 1
Synthesis of copolymers of 4-(14′-acryloxy-
3′-thia-1′-keto)tetradecyl phenyl boronic acid
and sodium 2-acrylamido-2-methyl-1-propanesulfonate
(AMPS) of varying compositions.

| Boronic acid | | | |
|---|---|---|---|
| monomer (g) | AMPS (g) | AIBN (mg) | Yield (g) |
| 2.0 | 0.27 | 22 | 1.15 |
| 0.5 | 0.17 | 7 | 0.4 |
| 0.5 | 0.4 | 10 | 0.4 |
| 0.5 | 1.0 | 15 | 0.8 |

### Example 29

### Synthesis of Poly{4-(14′-acryloxy-3′-thia-1′-keto)tetradecyl phenyl boronic acid-co-acrylic acid}

To a 25 ml, three necked, round bottomed flask were added 526 mg of 4-(14′-acryloxy-3′-thia-1′-keto)tetradecylphenyl boronic acid, 61 mg of acrylic acid, 5 mg of AIBN, and 5 ml of ethanol. The reaction mixture was bubbled with a slow stream of nitrogen for 30 minutes. While stirring the reaction mixture was heated to 65° C. and was stirred at this temperature under nitrogen atmosphere. The heating continued for 24 hr. Another batch of AIBN (6 mg) was added to the reaction mixture and the heating continued for a total period of 50 hr. After cooling down to room temperature, the reaction mixture was treated with 100 ml of diethyl ether and was stirred for 20 minutes. The solvent was removed by filtration. The residue was redissolved in 10 ml of THF and was precipitated from 1 00 ml of diethyl ether. The solvent was removed by filtration and residue was dried under vacuum yielding 225 mg of the polymer as an off white solid.

### Example 30

### Synthesis of Poly{4-(14′-acryloxy-3′-thia-1′-keto)tetradecyl phenyl boronic acid-co-sodium 4-styrene sulfonate}

Copolymers of this type were prepared in varying compositions of both the monomers. A general procedure for the synthesis of one of the copolymers is described here. Compositions of polymerization mixtures and yields of these copolymers are given in Table 2.

To a 250 ml, three necked, round bottomed flask were added 8.07 g of 4-(14′-acryloxy-3′-thia-1′-keto)tetradecylphenyl boronic acid, 2.62 g of sodium 4-styrene sulfonate, 85 ml of ethanol, 15 ml of deionized water, and 52 mg of AIBN. The reaction mixture was bubbled with a slow stream of nitrogen for 30 minutes. While stirring the reaction mixture was heated to 65° C. and was stirred at this temperature under nitrogen atmosphere. After heating for 24 hr another batch of AIBN (52 mg) was added. The heating continued for a total period of 50 hr. After cooling down to room temperature, the solvent was removed under reduced pressure. The residue was redissolved in 100 ml of THF and was precipitated from 500 ml of diethyl ether. This process of dissolution in THF and precipitation from diethyl ether was repeated two times. After filtration, the precipitate was dissolved in water and was dialyzed against deionized water for 24 hr using a 3,500 molecular weight cut-off membrane. The dialyzed polymer solution was dried in a forced air oven at 60° C. yielding 3.6 g of the polymer as an off-white solid.

TABLE 2
Synthesis of copolymers of 4-(14′-acryloxy-3′-
thia-1′-keto)tetradecyl phenyl boronic acid and sodium
4-styrene sulfonate (NaSS) of varying compositions.

| Boronic acid | | | |
|---|---|---|---|
| monomer (g) | NaSS (g) | AIBN (mg) | Yield (g) |
| 8.0 | 2.62 | 105 | 4.2 |
| 4.02 | 1.97 | 64 | 2.6 |
| 2.06 | 1.48 | 38 | 1.6 |
| 2.1 | 0.43 | 30 | 1.1 |

### Example 31

### Synthesis of Poly{4-(14′-acryloxy-3′-thia-1′-keto)tetradecyl phenyl boronic acid-co-(3-acrylamidopropyl)trimethylammonium chloride-co-acrylamide}

Terpolymers of this type were prepared by varying the amounts of different comonomers in the polymerization mixture. A general procedure for the synthesis of one of the terpolymers is described here. Compositions of polymerization mixtures and yields of these terpolymers are given in Table 3.

To a 250 ml, round bottomed flask were added 6.02 g of 4-(14′-acryloxy-3‘-thia-1’-keto)tetradecyl phenyl boronic acid, 5.9 g of (3-acrylamidopropyl)trimethylammonium chloride, 2.04 g of acrylamide, 70 mg of AIBN, and 50 ml of ethanol. The reaction mixture was bubbled with a slow stream of nitrogen for 30 minutes. While stirring the reaction mixture was heated to 65° C. and was stirred at this temperature under nitrogen atmosphere. After 48 hr another batch of AIBN (70 mg) was added and the heating continued for additional 24 hr. After cooling down to room temperature, the reaction mixture was treated with 700 ml of isopropanol and was stirred for 20 minutes. The solvent was removed by filtration. The residue was redissolved in 60 ml of THF and 5 ml of water. The resulting polymer solution was precipitated from 800 ml of isopropanol. The solvent was removed by filtration and the residue was dried under vacuum yielding 8.9 g of the polymer as an off white solid.

TABLE 3
Synthesis of terpolymers of 4-(14′-acryloxy-3′-
thia-1′-keto)tetradecyl phenyl boronic acid,
(3-acrylamidopropyl)trimethylammonium chloride (APTAC) and
acrylamide (AM) of varying compositions.

| Boronic acid | | | | |
|---|---|---|---|---|
| monomer (g) | APTAC (g) | AM (g) | AIBN (mg) | Yield (g) |
| 6.02 | 5.9 | 2.0 | 210 | 8.9 |
| 5.5 | 4.5 | 3.7 | 210 | 11 |
| 7.1 | 2.2 | 1.05 | 160 | 6.3 |

### Example 32

### Synthesis of Poly{4-(14′-acryloxy-3′-thia-1′-keto)tetradecyl phenyl boronic acid-co-(3-acrylamidopropyl)trimethylammonium chloride-co-sodium 2-acrylamido-2-methyl-1-propanesulfonate}

To a 250 ml, round bottomed flask were added 6.04 g of 4-(14′-acryloxy-3′-thia-1′-keto)tetradecyl phenyl boronic acid, 2.58 g of (3-acrylamidopropyl)-trimethylammonium chloride, 2.05 g of sodium 2-acrylamido-2-methyl-1-propane-sulfonate, 53 mg of AIBN, 72 ml of ethanol, and 17 ml of deionized water. The reaction mixture was bubbled with a slow stream of nitrogen for 30 minutes. While stirring the reaction mixture was heated to 65° C. and was stirred at this temperature under nitrogen atmosphere. After 48 hr of heating another batch of AIBN (53 mg) was added and the heating continued for additional 24 hr. After cooling down to room temperature, the reaction mixture was treated with 700 ml of isopropanol and was stirred for 20 minutes. The solvent was removed by filtration. The residue was redissolved in 60 ml of THF and 5 ml of water. The resulting polymer solution was precipitated from 800 ml of isopropanol. The solvent was removed by filtration and residue was dried under vacuum. Finally the polymer was dialyzed against deionized water for 24 hr and the dialyzed polymer solution was dried in a forced air oven at 60° C. yielding 6.7 g of the product as an off white solid.

### Example 33

### Synthesis of Poly{4-(14′-acyloxy-3′-thia-1′-keto)tetradecyl phenyl boronic acid-co-N-(3-sulfopropyl)-N-methacryloylamidopropyl-N,N-dimethylammonium betaine}

To a 250 ml, round bottomed flask were added 5 g of 4-(-14′-acryloxy-3′-thia-1′-keto)tetradecyl phenyl boronic acid, 2.32 g of N-(3-sulfopropyl)-N-methacryloylamido-propyl-N,N-dimethylammonium betaine, 37 mg of AIBN, 43 ml of ethanol, and 23 ml of deionized water. The reaction mixture was bubbled with a slow stream of nitrogen for 45 minutes. While stirring the reaction mixture was heated to 65° C. and was stirred at this temperature under nitrogen atmosphere. After 48 hr of heating, another batch of AIBN (48 mg) was added and the heating continued for additional 24 hr. The solvent was removed under reduced pressure and the residue was redissolved in 100 ml of THF. The resulting polymer solution was precipitated from 400 ml of diethyl ether. This dissolution in THF and reprecipitation from diethyl ether was repeated twice. After filtration the residue was dried under vacuum yielding 4.4 g of the polymer as an off white solid.

### Example 34

### Synthesis of Poly{4-(14′-acryloxy-3′-thia-1′-keto)tetradecyl phenyl boronic acid-co-N-(3-sulfopropyl)-N-methacryloylamidopropyl-N,N-dimethylammonium betaine-co-(3-acrylamidopropyl)trimethylammonium chloride}

To a 250 ml, round bottomed flask were added 4.4 g of 4-(−14′-acryloxy-3′-thia-1′-keto)tetradecyl phenyl boronic acid, 2.70 g of N-(3-sulfopropyl)-N-methacryloylamido-propyl-N,N-dimethylammonium betaine, 1.35 g of (3-acrylamidopropyl)trimethylammonium chloride, 42 mg of AIBN, 48 ml of ethanol, and 10 ml of deionized water. The reaction mixture was bubbled with a slow stream of nitrogen for 45 minutes. While stirring the reaction mixture was heated to 65° C. and was stirred at this temperature under nitrogen atmosphere. After 48 hr of heating another batch of AIBN (52 mg) was added and the heating continued for additional 24 hr. The solvent was removed under reduced pressure and the residue was redissolved in 100 ml of THF and 3 ml of deionized water. The resulting polymer solution was precipitated from 400 ml of diethyl ether. This dissolution in THF and reprecipitation from diethyl ether was repeated twice. The solvent was removed by filtration and residue was dried under vacuum yielding 6.4 g of the polymer as an off white solid.

### Example 35

### Synthesis of Poly{4-(14′-acryloxy-3′-thia-1′-keto)tetradecyl phenyl boronic acid-co-potassium 3-sulfopropyl acrylate}

To a 100 ml, three necked, round bottomed flask were added 2.0 g of 4-(14′-acryloxy-3′-thia-1′-keto)tetradecyl phenyl boronic acid, 0.75 g of potassium 3-sulfopropyl acrylate, 22 ml of ethanol, 5 ml of deionized water, and 17 mg of AIBN. The reaction mixture was bubbled with a slow stream of nitrogen for 30 minutes. While stirring the reaction mixture was heated to 65° C. and was stirred at this temperature under nitrogen atmosphere. After heating for 24 hr another batch of AIBN (15 mg) was added. The heating continued for a total period of 50 hr. After cooling down to room temperature, the solvent was removed under reduced pressure. The residue was redissolved in 15 ml of a solvent system containing THF, methanol, and deionized water (9:8:2, v/v) and was precipitated from 200 ml of diethyl ether. This process of dissolution and precipitation was repeated two times. After filtration, the precipitate was dissolved in water and was dialyzed against deionized water for 24 hr using a 3,500 molecular weight cut-off membrane. The dialyzed polymer solution was dried in a forced air oven at 60° C. yielding 1.1 g of the polymer as an off-white solid.

### Example 36

### Synthesis of Poly{4-(14′-acryloxy-3′-thia-1′-keto)tetradecyl phenyl boronic acid-co-4-vinylbenzyl phosphonic acid}

To a 100 ml, three-necked, round-bottomed flask were added 3.04 g of 4-(14′-acryloxy-3′-thia-1′-keto)tetradecyl phenyl boronic acid, 0.94 g of 4-vinylbenzyl phosphonic acid, 25 ml of ethanol, 4 ml of deionized water, and 20 mg of AIBN. The reaction mixture was bubbled with a slow stream of nitrogen for 30 minutes. While stirring the reaction mixture was heated to 65° C. and was stirred at this temperature under nitrogen atmosphere. After heating for 24 hr another batch of AIBN (20 mg) was added. The heating continued for a total period of 48 hr. After cooling down to room temperature, the solvent was removed under reduced pressure. The residue was redissolved in 15 ml of THF and 1 ml of deionized water and was precipitated from 200 ml of diethyl ether. This process of dissolution and precipitation was repeated two times. After filtration, the precipitate was dissolved in weakly alkaline water (pH=8.0) and was dialyzed against deionized water for 24 hr using a 3,500 molecular weight cut-off membrane. The dialyzed polymer solution was dried in a forced air oven at 60° C. yielding 1.6 g of the polymer as an off-white solid.

### Example 37

### Synthesis of Poly{4-(14′-acryloxy-3′-thia-1′-keto)tetradecyl phenyl boronic acid-co-poly(ethyleneglycol)methyl ether acrylate}

To a 100 ml, three necked, round bottomed flask were added 2.5 g of 4-(14′-acryloxy-3′-thia-1′-keto)tetradecyl phenyl boronic acid, 1.1 g of poly(ethylene glycol)methyl ether acrylate (average MW=454), 30 ml of ethanol, and 17 mg of AIBN. The reaction mixture was bubbled with a slow stream of nitrogen for 30 minutes. While stirring the reaction mixture was heated to 65° C. and was stirred at this temperature under nitrogen atmosphere. After heating for 24 hr another batch of AIBN (16 mg) was added. The heating continued for a total period of 48 hr. After cooling down to room temperature, the solvent was removed under reduced pressure. The residue was redissolved in 15 ml of THF and 1 ml of deionized water and was precipitated from 200 ml of diethyl ether. This process of dissolution and precipitation was repeated two times. After filtration, the precipitate was dissolved in water and was dialyzed against deionized water for 24 hr using a 3,500 molecular weight cut-off membrane. The dialyzed polymer solution was dried in a forced air oven at 60° C. yielding 1.1 g of the polymer as an off-white solid.

### Example 38

### Synthesis of Poly{4-(14′-methacryloxy-3′-thia-1′-keto)tetradecyl phenyl boronic acid-co-N,N-dimethyl acrylamide}

To a 100 ml, three-necked, round-bottomed flask were added 1.9 g of 4-(14′-methacryloxy-3′-thia-1′-keto)tetradecyl phenyl boronic acid, 1.26 g of N,N-dimethyl acrylamide, 20 ml of ethanol, and 18 mg of AIBN. The reaction mixture was bubbled with a slow stream of nitrogen for 30 minutes. While stirring the reaction mixture was heated to 65° C. and was stirred at this temperature under nitrogen atmosphere. After heating for 24 hr another batch of AIBN (16 mg) was added. The heating continued for a total period of 40 hr. After cooling down to room temperature, the solvent was removed under reduced pressure. The residue was redissolved in 15 ml of THF and 1 ml of deionized water and was precipitated from 200 ml of diethyl ether. This process of dissolution and precipitation was repeated three times. After filtration, the residue was dried at 45° C. under vacuum yielding 1.5 g of the polymer as off white solid.

### Example 39

### Synthesis of Poly{4-(14′-methacryloxy-3′-thia-1′-keto)tetradecyl phenyl boronic acid-co-sodium 2-acrylamido-2-methyl-1-propanesulfonate}

To a 50 ml, three necked, round bottomed flask were added 2.05 g of 4-(14′-methacryloxy-3′-thia-1′-keto)tetradecyl phenyl boronic acid, 700 mg of sodium 2-acrylamido-2-methyl-1-propanesulfonate, 16 mg of AIBN, and 25 ml of ethanol. The reaction mixture was bubbled with a slow stream of nitrogen for 30 minutes. While stirring the reaction mixture was heated to 65° C. and was stirred at this temperature under nitrogen atmosphere. The heating continued for 24 hr. Another batch of AIBN (16 mg) was added and heating continued for a total period of 48 hr. The solvent was removed under reduced pressure. The residue was redissolved in 15 ml of a solvent system containing THF, methanol, and deionized water (9:8:2, v/v) and was precipitated from 200 ml of diethyl ether. This process of dissolution and precipitation was repeated twice. After filtration, the precipitate was dissolved in water and was dialyzed against deionized water for 24 hr using a 3,500 molecular weight cut-off membrane. The dialyzed polymer solution was dried in a forced air oven at 60° C. yielding 0.9 g of the polymer as an off-white solid.

### Example 40

### Synthesis of Poly{4-(14′-methacryloxy-3′-thia-1′-keto)tetradecyl phenyl boronic acid-co-sodium 4-styrene sulfonate}

To a 250 ml, three necked, round bottomed flask were added 2.02 g of 4-(14′-methacryloxy-3′-thia-1′-keto)tetradecyl phenyl boronic acid, 0.64 g of sodium 4-styrene sulfonate, 20 ml of ethanol, 4.5 ml of deionized water, and 14 mg of AIBN. The reaction mixture was bubbled with a slow stream of nitrogen for 30 minutes. While stirring the reaction mixture was heated to 65° C. and was stirred at this temperature under nitrogen atmosphere. After heating for 24 hr, 16 mg of AIBN was added. The heating continued for a total period of 50 hr. After cooling down to room temperature, the solvent was removed under reduced pressure. The residue was redissolved in 20 ml of a solvent system containing THF, methanol, and deionized water (9:8:2, v/v) and was precipitated from 200 ml of diethyl ether. This process of dissolution and precipitation was repeated twice. After filtration, the precipitate was dissolved in water and was dialyzed against deionized water for 24 hr using a 3,500 molecular weight cut-off membrane. The dialyzed polymer solution was dried in a forced air oven at 60° C. yielding 1.1 g of the polymer as an off-white solid.

### Example 41

### Synthesis of Poly{4-(9′-acryloxy-3′-thia-1′-keto)nonyl phenyl boronic acid-co-sodium 2-acrylamido-2-methyl-1-propanesulfonate}

To a 100 ml, three-necked, round bottomed flask were added 1.86 g of 4-(9′-acryloxy-3′-thia-1′-keto)nonyl phenyl boronic acid, 810 mg of sodium 2-acrylamido-2-methyl-1-propanesulfonate, 13 mg of AIBN, 20 ml of ethanol. The reaction mixture was bubbled with a slow stream of nitrogen for 30 minutes. While stirring the reaction mixture was heated to 65° C. and was stirred at this temperature under nitrogen atmosphere. After 24 hr of heating another batch of 15 mg of AIBN was added and the heating continued for a total period of 48 hr. The solvent was removed under reduced pressure. The residue was redissolved in 15 ml of THF and 1 ml of deionized water and was precipitated from 200 ml of diethyl ether. This process of dissolution and precipitation was repeated twice. After filtration, the precipitate was dissolved in water and was dialyzed against deionized water for 24 hr using a 3,500 molecular weight cut-off membrane. The dialyzed polymer solution was dried in a forced air oven at 60° C. yielding 1.2 g of the polymer as an off-white solid.

### Example 42

### Synthesis of Poly{4-(12′-acryloxy-3′-thia-1′-keto)dodecyl phenyl boronic acid-co-sodium 2-acrylamido-2-methyl-1-propanesulfonate}

To a 100 ml, three necked, round bottomed flask were added 1.5 g of 4-(12′-acryloxy-3′-thia-1′-keto)dodecyl phenyl boronic acid, 580 mg of sodium 2-acrylamido-2-methyl-1-propanesulfonate, 13 mg of AIBN, 17 ml of ethanol, and 3.5 ml of deionized water. The reaction mixture was bubbled with a slow stream of nitrogen for 30 minutes. While stirring the reaction mixture was heated to 65° C. and was stirred at this temperature under nitrogen atmosphere. After heating for 24 hr another batch of AIBN (13 mg) was added and heating continued for a total period of 40 hr. The solvent was removed under reduced pressure. The residue was redissolved in 15 ml of THF and 1 ml of deionized water and was precipitated from 200 ml of diethyl ether. This process of dissolution and precipitation was repeated twice. After filtration, the precipitate was dissolved in water and was dialyzed against deionized water for 24 hr using a 3,500 molecular weight cut-off membrane. The dialyzed polymer solution was dried in a forced air oven at 60° C. yielding 1.1 g of the polymer as an off-white solid.

### Example 43

### Synthesis of Poly{4-(14′-acryloxy-3′-thia-1′-keto)tetradecyl-3-fluorophenyl boronic acid}

To a 50 ml, round bottomed flask were added 1.8 g 4-(14′-acryloxy-3′-thia-1′-keto)tetradecyl-3-fluorophenyl boronic acid, 15 mg of AIBN, and 10 ml of ethanol. The reaction mixture was bubbled with a slow stream of nitrogen for 30 minutes. While stirring the reaction mixture was heated to 65° C. and was stirred at this temperature under nitrogen atmosphere. The heating continued for 48 hr. After cooling down to room temperature, the reaction mixture was treated with 50 ml of diethyl ether and was stirred for 20 minutes. The solvent was removed by filtration. The residue was redissolved in 10 ml of THF and was precipitated from 100 ml of diethyl ether. The solvent was removed by filtration and residue was dried under vacuum yielding 820 mg of the polymer as an off white solid.

### Example 44

### Synthesis of Poly{4-(14′-acryloxy-3′-thia-1′-keto)tetradecyl-3-fluoro phenyl boronic acid-co-(3-acrylamidopropyl)trimethylammonium chloride}

To a 50 ml, round bottomed flask were added 685 mg of 4-(14′-acryloxy-3′-thia-1′-keto)tetradecyl-3-fluoro phenyl boronic acid, 600 mg of (3-acrylamidopropyl) trimethylammonium chloride, 7 mg of AIBN, and 8 ml of ethanol. The reaction. mixture was bubbled with a slow stream of nitrogen for 30 minutes. While stirring the reaction mixture was heated to 65° C. and was stirred at this temperature under nitrogen atmosphere. The heating continued for 48 hr. Another batch of AIBN (6 mg) was added and the heating continued for additional 24 hr. After cooling down to room temperature, the reaction mixture was treated with 50 ml of diethyl ether and was stirred for 20 minutes. The solvent was removed by filtration. The residue was redissolved in 10 ml of THF and 1 ml of deionized water and was precipitated from 100 ml of diethyl ether. This dissolution and reprecipitation procedure was repeated twice. The solvent was removed by filtration and residue was dried under vacuum yielding 900 mg of the polymer as an off white solid.

### Example 45

### Synthesis of Poly{12-(4′-vinyl)phenoxy dodecyl(4″-boronato)benzoate}

To a 50 ml, round-bottomed flask were added 800 mg of 12-(4′-vinyl)phenoxy dodecyl-(4″-boronato)benzoate, 10 ml of 1,4-dioxane, and 10 mg of AIBN. The reaction mixture was bubbled with a slow stream of nitrogen for 30 minutes. While stirring the reaction mixture was heated to 65° C. and was stirred at this temperature under nitrogen atmosphere. The heating continued for 24 hr. Another batch of AIBN (6 mg) was added and the heating continued for additional 48 hr. After cooling down to room temperature, the reaction mixture was treated with 100 ml of diethyl ether and was stirred for 20 minutes. The solvent was removed by filtration. The residue was redissolved in 5 ml of THF and was precipitated from 50 ml of methanol. This dissolution and reprecipitation procedure was repeated one more time. The solvent was removed by filtration and residue was dried under vacuum at 60° C. yielding 200 mg of the polymer as an off white solid.

### Example 46

### Synthesis of Poly{6-(4′-vinyl)phenoxy hexyl (4″-boronato)benzoate}

To a 50 ml, round-bottomed flask were added 1 g of 6-(4′-vinyl)phenoxy hexyl (4″-boronato)benzoate, 10 ml of 1,4-dioxane, and 12 mg of AIBN. The reaction mixture was bubbled with a slow stream of nitrogen for 30 minutes. The reaction mixture was heated to 65° C. and was stirred at this temperature under nitrogen atmosphere. The heating continued for 24 hr. Another batch of AIBN (10 mg) was added and the heating continued for additional 48 hr. After cooling down to room temperature, the reaction mixture was treated with 100 ml of diethyl ether and was stirred for 20 minutes. The solvent was removed by filtration. The residue was redissolved in 5 ml of THF and was precipitated from 50 ml of methanol. This dissolution and reprecipitation procedure was repeated one more time. The solvent was removed by filtration and residue was dried under vacuum at 60° C. yielding 250 mg of the polymer as an off white solid.

### Example 47

### Poly{N-(3-boronato)phenyl 10-undecenamide-co-maleic anhydride}

To a 250 ml, three-necked, round-bottomed flask were added 9.0 g of N-(3-boronato)phenyl 10-undecenamide, 3.14 g of maleic anhydride, 60 ml of 1,4-dioxane, and 260 mg of AIBN. The reaction mixture was bubbled with a slow stream of nitrogen for 30 minutes. While stirring the reaction mixture was heated to 65° C. and was stirred at this temperature under nitrogen atmosphere. After 24 hr of heating another batch of AIBN (140 mg) was added and the heating continued for additional 48 hr. After cooling down to room temperature, the reaction mixture was precipitated from 500 ml of diethyl ether. After filtration the precipitate was redissolved in 30 ml of THF and was reprecipitated from 300 ml of diethyl ether. This process was repeated one more time and the residue was dried under reduced pressure yielding 6.0 g of the polymer as an off-white solid.

### Example 48

### Synthesis of Poly{N-(3′-boronato)phenyl (11-acrylamido)undecylamide}

To 25 ml, round-bottomed flask were added 500 mg of N-(3′-boronato)phenyl (11-acrylamido)-undecylamide, 5 mg of AIBN, and 5 ml of ethanol. The reaction mixture was bubbled with a slow stream of nitrogen for 30 minutes. While stirring the reaction mixture was heated to 65° C. and was stirred at this temperature under nitrogen atmosphere. The heating continued for 48 hr. After cooling down to room temperature, the reaction mixture was precipitated from 100 ml of diethyl ether. After filtration the precipitate was redissolved in 5 ml of ethanol and was reprecipitated from 100 ml of diethyl ether. After filtration, the residue was dried under reduced pressure yielding 400 mg of the polymer as an off-white solid.

### Example 49

### Synthesis of Poly{N-(3′-boronato)phenyl (11-acrylamido)undecylamide-co-N,N-diethyl acrylamide}

To 25 ml, round bottomed flask were added 500 mg of N-(3′-boronato)phenyl (11-acrylamido)-undecylamide, 510 mg of N,N-diethyl acrylamide, 20 mg of AIBN, and 10 ml of ethanol. The reaction mixture was bubbled with a slow stream of nitrogen for 30 minutes. While stirring the reaction mixture was heated to 65° C. and was stirred at this temperature under nitrogen atmosphere. The heating continued for 48 hr. After cooling down to room temperature, the reaction mixture was precipitated from 100 ml of diethyl ether. After filtration the precipitate was redissolved in 5 ml of ethanol and was reprecipitated from 100 ml of diethyl ether. After filtration, the residue was dried under reduced pressure yielding 600 mg of the polymer as an off-white solid.

### Example 50

### Synthesis of Poly{N-(3′-boronato)phenyl (11-acrylamido)undecylamide-co-N-butyl acrylamide}

To a 25 ml, round-bottomed flask were added 500 mg of N-(3′-boronato) phenyl (11-acrylamido)-undecylamide, 510 mg of N-butyl acrylamide, 20 mg of AIBN, and 10 ml of ethanol. The reaction mixture was bubbled with a slow stream of nitrogen for 30 minutes. While stirring the reaction mixture was heated to 65° C. and was stirred at this temperature under nitrogen atmosphere. The heating continued for 48 hr. After cooling down to room temperature, the reaction mixture was precipitated from 100 ml of diethyl ether. After filtration the precipitate was redissolved in 10 ml of ethanol and was reprecipitated from 100 ml of diethyl ether. After filtration, the residue was dried under reduced pressure yielding 750 mg of the polymer as an off-white solid.

### Example 53

### Synthesis of Poly{11-acryloxy undecyl (4′-boronato) benzoate-co-acrylic acid)

To a 25 ml, round-bottomed flask were added 890 mg of 11-acryloxy undecyl (4′-boronato) benzoate, 110 mg of acrylic acid, 5 mg of AIBN, and 5 ml of ethanol. The reaction mixture was bubbled with a slow stream of nitrogen for 30 minutes. While stirring the reaction mixture was heated to 65° C. and was stirred at this temperature under nitrogen atmosphere. The heating continued for 48 hr. After cooling down to room temperature, the reaction mixture was precipitated from 50 ml of hexane. After filtration the residue was washed with 100 ml of hexane:ethyl acetate (6:4, v/v) mixture and was dried under reduced pressure yielding 300 mg of the polymer as an off-white solid.

### Example 54

### Synthesis of Poly{11-acryloxy undecyl (4′-boronato) benzoate-co-sodium 2-acrylamido-2-methyl-1-propanesulfonate}

To a 50 ml, round bottomed flask. were added 1.0 g of 11-acryloxy undecyl (4′-boronato) benzoate, 147 mg of sodium 2-acrylamido-2-methyl-1-propanesulfonate, 6 mg of AIBN, and 8 ml of ethanol. The reaction mixture was bubbled with a slow stream of nitrogen for 30 minutes. While stirring the reaction mixture was heated to 65° C. and was stirred at this temperature under nitrogen atmosphere. Aliquots of AIBN (6 mg each) were added after 24 and 48 hrs. The heating continued for a total period of 72 hr. After cooling down to room temperature, the reaction mixture was treated with 30 ml of hexane and was stirred for 20 minutes. The solvent was removed by filtration. The residue was washed with 40 ml of hexane followed by deionized water (2×40 ml). Subsequently, the residue was dried under vacuum yielding 800 mg of the polymer as an off white solid.

### Example 55

### Synthesis of Poly{11-acryloxy undecyl (4′-boronato) benzoate-co-(3 acrylamido-propyl)trimethylammonium chloride-co-acrylamide}

To a 50 ml, round bottomed flask were added 850 mg of 11-acryloxy undecyl (4′-boronato) benzoate, 250 mg of (3-acrylamidopropyl)trimethylammonium chloride, 100 mg of acrylamide, 6 mg of AIBN, and 7 ml of ethanol. The reaction mixture was bubbled with a slow stream of nitrogen for 30 minutes. The reaction mixture was heated to 65° C. and was stirred at this temperature under nitrogen atmosphere. The heating continued for 48 hr. After cooling down to room temperature, the reaction mixture was treated with 40 ml of isopropanol and was stirred for 20 minutes. The solvent was removed by filtration. The residue was washed with 40 ml of ethyl acetate followed by 40 ml of isopropanol. The washed residue was dried under vacuum yielding 880 g of the polymer as an off white solid.

### Example 56

### Synthesis of Poly{11-acryloxy undecyl (4′-boronato) benzoate-co-N-vinyl pyrrolidone}

To a 25 ml, round-bottomed flask were added 1.0 g of 11-acryloxy undecyl (4′-boronato) benzoate, 190 mg of N-vinyl pyrrolidone, 6 mg of AIBN, and 6 ml of ethanol. The reaction mixture was bubbled with a slow stream of nitrogen for 30 minutes. While stirring the reaction mixture was heated to 65° C. and was stirred at this temperature under nitrogen atmosphere. The heating continued for 48 hr. After cooling down to room temperature, the reaction mixture was precipitated from 50 ml of hexane. After filtration the residue was washed with ethyl acetate (3×40 ml) and was dried under reduced pressure yielding 860 mg of the polymer as an off-white solid.

### Example 57

### Synthesis of Poly{11-acryloxy undecyl (4′-boronato) benzoate-co-N-(3-sulfopropyl)-N-methacryloylamidopropyl-N,N-dimethylammonium betaine}

To a 25 ml, round bottomed flask were added 800 mg of 11-acryloxy undecyl (4′-boronato) benzoate, 420 mg of N-(3-sulfopropyl)-N-methacryloylamidopropyl-N,N-dimethylammonium betaine, 6 mg of AIBN, and 6 ml of ethanol. The reaction mixture was bubbled with a slow stream of nitrogen for 30 minutes. While stirring the reaction mixture was heated to 65° C. and was stirred at this temperature under nitrogen atmosphere. The heating continued for 48 hr. After cooling down to room temperature, the reaction mixture was precipitated from 50 ml of hexane. After filtration the residue was washed with ethyl acetate (3×40 ml) followed by 40 ml of deionized water. The washed residue was dried under reduced pressure yielding 900 mg of the polymer as an off-white solid.

### Example 58

### Synthesis of Poly{11-acryloxy undecyl (4′-boronato) benzoate-co-acrylic acid}

To a 25 ml, round bottomed flask were added 1.0 g of 11-acryloxy undecyl (4′-boronato) benzoate, 176 mg of (3-acrylamidopropyl)trimethylammonium chloride, 6 mg of AIBN, and 5 ml of ethanol. The reaction mixture was bubbled with a slow stream of nitrogen for 30 minutes. While stirring the reaction mixture was heated to 65° C. and was stirred at this temperature under nitrogen atmosphere. The heating continued for 48 hr. After cooling down to room temperature, the reaction mixture was precipitated from 50 ml of hexane. After filtration the residue was washed with 100 ml of a hexane:ethyl acetate (6:4, v/v) mixture followed by 50 ml of deionzied water. The residue was dried under reduced pressure yielding 700 mg of the polymer as an off-white solid.

### Example 59

### Synthesis of Comb Copolymer of 4-(14′-acryloxy-3′-thia-1′-keto)tetradecyl phenyl boronic acid and 3-(acrylamidopropyl)trimethylammonium chloride

Synthesis of this copolymer structure was accomplished by reacting an amino terminated macromer of 3-(acrylamidopropyl)trimethylammonium chloride with a reactive copolymer of 4-(14′-acryloxy-3′-thia-1′-keto)tetradecyl phenyl boronic acid and N-acryloxy succinimide. Scheme 1 illustrates the synthesis of this graft copolymer. Overall synthesis of this copolymer involves the following three steps.

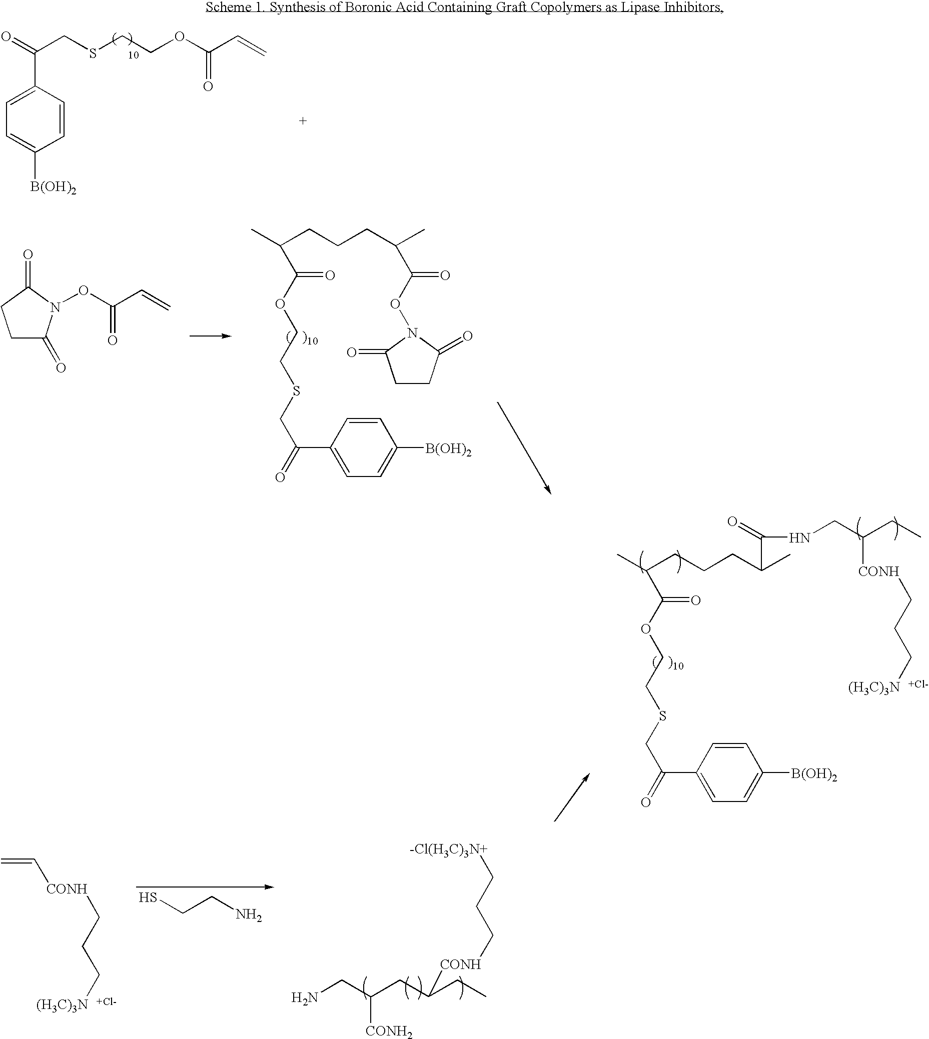

59a. Synthesis of amine terminated poly{3-(acrylamidopropyl)trimethylammonium chloride} macromer. To a 500 ml, three-necked, round-bottomed flask were added 15.0 g of (3-acrylamidopropyl)trimethylammonium chloride, 119 mg of AIBN, 1.36 g of cystamine hydrochloride, and 75 ml of ethanol. The reaction mixture was bubbled with a slow stream of nitrogen for 30 minutes. While stirring the reaction mixture was heated to 65° C. and was stirred at this temperature under nitrogen atmosphere. The heating continued for 24 hr. After cooling down to room temperature, a solution of 750 mg of potassium hydroxide in 75 ml of methanol was added slowly to the reaction mixture. After stirring for 15 minutes, the solution was poured into 600 ml of diethyl ether. The mixture was stirred for 20 minutes and was filtered. The residue was redissolved in 50 ml of methanol and was reprecipitated from 500 ml of diethyl ether. After filtration the residue was dried under reduced pressure at 35° C. yielding 12.5 g of the polymer as an off-white solid.

59b. Synthesis of Poly{4-(14′-acryloxy-3′-thia-1′-keto)tetradecyl phenyl boronic acid-co-N-acryloxy succinimide}. To a 100 ml, three-necked, round-bottomed flask were added 3.0 g of 4-(14′-acryloxy-3′-thia-1′-keto)tetradecyl phenyl boronic acid, 530 mg of N-acryloxy succinimide, 35 ml of DMF, and 30 mg of AIBN. The reaction mixture was bubbled with a slow stream of nitrogen for 30 minutes. While stirring the reaction mixture was heated to 65° C. and was stirred at this temperature under nitrogen atmosphere for 36 hr. After cooling down to room temperature, the solution was kept under nitrogen atmosphere.

59c. Synthesis of Poly 4-(-14′-acryloxy-3′-thia-1′-keto)tetradecyl phenyl boronic acid-co-3-(acrylamidopropyl)trimethylammonium chloride} graft copolymer. To the DMF solution of Poly{4-(14′-acryloxy-3′-thia-1′-keto)tetradecyl phenyl boronic acid-co-N-acryloxy succinimide} prepared in step 59b was added 2 g of amine-terminated poly{3-(acrylamidopropyl)trimethylammonium chloride} macromer dissolved in 20 ml of DMSO. The reaction mixture was stirred at 40° C. for an hour and 60 ml of DMSO were added to the reaction mixture. The reaction mixture was subsequently stirred at 40° C. for an additional 30 hr. After cooling down to room temperature, the solution was poured into 800 ml of diethyl ether and stirred for 20 minutes. After filtration, the residue was dissolved in 200 ml of deionized water. It was dialyzed against deionized water for 48 hr using a 3500 molecular weight cut-off membrane. The dialyzed solution was dried at 60° C. in a forced air oven yielding 3.3 g of the polymer as off-white solid.

### Example 60

### Synthesis of Poly{6-(4′-vinyl)phenoxy hexyl (4″-boronato)benzoate-co-sodium 4-styrene sulfonate} block copolymer

This block copolymer containing segments of poly{6-(4′-vinyl)phenoxy hexyl (4″-boronato)benzoate and poly(sodium 4-styrene sulfonate) chains were prepared by nitroxide mediated living free radical polymerization. Scheme 2 illustrates the synthesis of this block copolymer, which was accomplished in two steps. 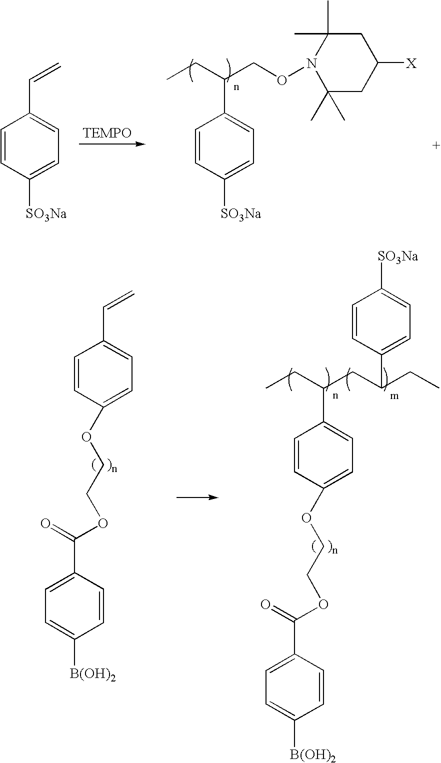
Scheme 2. Synthesis of boronic acid functionalized block copolymers as lipase inhibitors

60a. Synthesis of nitroxide terminated poly(sodium 4-styrene sulfonate) macromer. To a 50 ml, round bottomed flask were added 2.06 g of sodium 4-styrene sulfonate, 82 mg of AIBN, 172 mg of 4-hydroxy TEMPO, 12 ml of ethylene glycol, and 5 ml of water. The reaction mixture was bubbled with a slow stream of nitrogen for 30 minutes. While stirring the reaction mixture was heated to 130° C. and was stirred at this temperature under nitrogen atmosphere. The heating continued for 72 hr. After cooling down to room temperature, the reaction mixture was precipitated from 100 ml of THF. After filtration the residue was dissolved in 10 ml of deionized water and precipitated from 80 ml of isopropanol. After filtration the residue was dried under reduced pressure at 40° C. yielding 1.2 g of the polymer as an off-white solid.

60b. Synthesis of Poly {6-(4′-vinyl)phenoxy hexyl (4″-boronato)benzoate-co-sodium 4-styrene sulfonate} block copolymer. To a 50 ml, round bottomed flask were added 225 mg of nitroxide terminated poly(sodium 4-styrene sulfonate) macromer, 400 mg of 6-(4′-vinyl)phenoxy hexyl (4″-boronato)benzoate, and 10 ml of DMSO. The reaction mixture was bubbled with a slow stream of nitrogen for 30 minutes. While stirring the reaction mixture was heated to 130° C. and was stirred at this temperature under nitrogen atmosphere. The heating continued for 24 hr. After cooling down to room temperature, the reaction mixture was precipitated from 100 ml of diethyl ether. After filtration the residue was dissolved in 5 ml of methanol and precipitated from 100 ml of diethyl ether. After filtration the residue was dried under reduced pressure at 40° C. yielding 300 mg of the polymer as an off-white solid.

### Example 61

Results of In Vitro Inhibition of Pancreatic Lipase Using Polymeric Boronic Acid Based Enzyme Inhibitors

An in vitro assay of pancreatic lipase activity was used to measure the efficacy of lipase inhibitory compounds. Porcine pancreatic lipase (23 units/milliliters) was incubated for 4 hours at 37° C. with 72 mM triglyceride (as an olive oil/gum arabic emulsion) in 5.5 milliliters of a 300 mM BES buffer, pH 7.0, containing 10 mM CaCl₂, 109 mM NaCl, and 8 mM sodium taurocholate. The reaction was stopped by acidification with HCl and the lipids were extracted by the method disclosed in Folch, et al., J. Biol. Chem. 226:497 (1957) prior to analysis by HPLC. An aliquot of the chloroform layer was evaporated and reconstituted in hexane, and the sample was analyzed on a Waters Alliance 2690 HPLC with a Sedex 55 Evaporative Light Scattering detector utilizing a YMC PVA Sil 3×50 millimeter column. The mobile phase consisted of hexane and methyl t-butyl ether delivered in a linear gradient at a flow rate of 0.5 milliliters/minute. External standards were utilized for quantification of triglycerides, diglycerides, and fatty acids, and the percent lipolysis was determined. For evaluation of lipase inhibitor efficacy, compounds were dissolved in DMSO or another appropriate solvent and added directly to the assay mixture prior to incubation. Inhibition was determined relative to a control incubation and IC₅₀ values were calculated from a plot of % inhibition vs. inhibitor concentration. IC₅₀ values are shown in Table 4. As can be seen, the polymers tested are effective inhibitors of lipase. Polymers are referenced by example number, as shown above.

TABLE 4

| Polymer | IC₅₀ (μg/g fat) |
|---|---|
| Example 18 | 320 |
| Example 24 | 320 |
| Example 25 | 10 |
| Example 26 | 29 |
| Example 27 | 16 |
| Example 31 | 270 |
| Example 35 | 8.1 |
| Example 40 | 39 |
| Example 42 | 8.2 |
| Example 44 | 48 |
| Example 46 | 15 |
| Example 53 | 50 |
| Example 54 | 3.2 |
| Example 55 | 39 |
| Example 59 | 10 |
| Example 28 (as shown in Table 1) | |
| Entry 1 | 7.0 |
| Entry 2 | 10 |
| Example 30 (as shown in Table 2) | |
| Entry 1 | 4.4 |
| Entry 2 | 7.8 |
| Entry 3 | 11 |

### Example 62

Results of In Vitro Inhibition of Pancreatic Lipase Using Polymeric Boronic Acid Based Enzyme Inhibitors

Compounds were evaluated in rats to determine their in vivo potency in inhibiting fat absorption through lipase inhibition. Rats were acclimated to the facility for approximately 1 week in individual wire-bottom cages and provided a standard chow diet and water ad libitum. Rats were then randomly assigned to groups of 4. They were gavaged at (7-8 AM) with 4 milliliters olive oil emulsified with gum arabic, with or without drug following an 18 hour fast. Test compounds were dissolved in DMSO or deionized water. Drug solutions were mixed thoroughly in the olive oil emulsion just prior to administration. After 8 hours, rats were euthanized with CO₂ and the intestines were removed. The intestinal contents were harvested from the lower half of the small intestine and the cecum. Contents were placed in separate, pre-weighed, 15 milliliter conical screw cap tubes in a (dry ice/alcohol bath) to maintain freezing temperature until the final freeze of all samples. Samples were stored at −80° C. until lyophilization. Samples were freeze-dried and ground, then analyzed for triglyceride and fatty acid.

A 20 milligram aliquot of each sample was weighed and transferred to a 15 milliliters conical tube. 3 milliliters of hexane were added to each tube, which were capped and vortexed for 15 seconds at high speed. 3 milliliters of 1 N HCl were added and the samples were then subjected to wrist-action shaking for 1 hour. Samples were then centrifuged for 5 minutes at 3500 rpm and the hexane layer was collected. An aliquot of the hexane layer was diluted in hexane and analyzed for triglyceride, diglyceride and fatty acid by HPLC as described above.

The data was expressed as follows. The milligrams of intestinal contents that was extracted and the total number of milligrams collected were recorded. The milligrams/milliliters values obtained from the HPLC analysis were entered. The individual lipid components were calculated and expressed as total milligrams recovered. Dose units are expressed as the milligrams of drug per gram of oil administered to each rat. The ED₅₀'s were determined by extrapolating the dose value at half the maximum obtainable triglyceride recoverable in the assay. The results are shown in Table 5. As can be seen, the polymers are effective in inhibiting lipolysis in vivo. Polymers are referenced by example number, as shown above.

TABLE 5

| | In vivo Infusion Assay in Rats |
|---|---|
| Polymer | ED₅₀ (mg/kg body wt) or estimate |
| Example 28, entry 3 | 50 |
| Example 30, entry 2 | 50 |
| Example 30, entry 4 | 60 |
| Example 30, entry 3 | 75 |
| Example 31, entry 2 | 420 |
| Example 37 | 75 |
| Example 40 | 60 |
| Example 41 | 75 |
| Example 42 | 75 |

While this invention has been particularly shown and described with references to preferred embodiments thereof, it will be understood by those skilled in the art that various changes in form and details may be made therein without departing from the scope of the invention encompassed by the appended claims.

1. A polymer substituted with at least one group represented by Structural Formula (I) or (II): 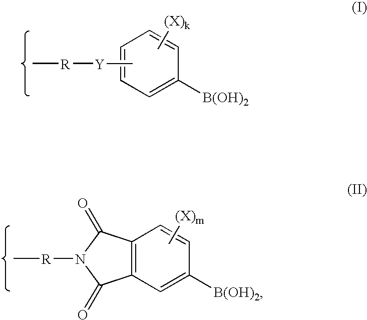
wherein:
R is a C6-C30 hydrocarbylene group optionally interrupted by one or more heteroatoms selected from the group consisting of NH, S, and O;
each X is independently a substituted or unsubstituted alkyl group, an electron withdrawing group, or an electron donating group meta to the boronic acid moiety;
Y is —C(O)Z-, -ZC(O)— or —S(CH₂)ₙ—;
Z is a bond, CH₂S, S, NH, or O;
m is an integer from 0 to 3;
k is an integer from 0 to 4; and
n is an integer from 0 to 5.

2. The polymer of claim 1, wherein each X is independently —H, a halogen, nitrile, ester or sulfone.

3. The polymer of claim 2, wherein said polymer is substituted with at least one group represented by Structural Formula (III) or (IV): 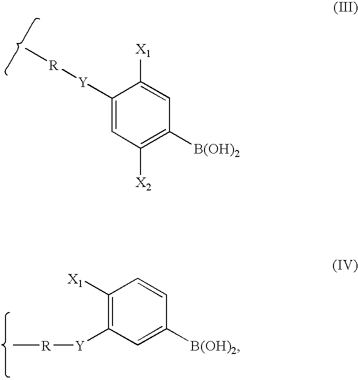
wherein X₁ and X₂ are each independently —H, a halogen or nitrile; and Y is —C(O)Z- or -ZC(O)—.

4. The polymer of claim 3, wherein said polymer is substituted with at least one group represented by Structural Formula (V):

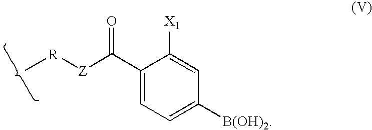

5. The polymer of claim 4, wherein said polymer is substituted with at least one group represented by Structural Formula (VI), (VII), or (VIII): 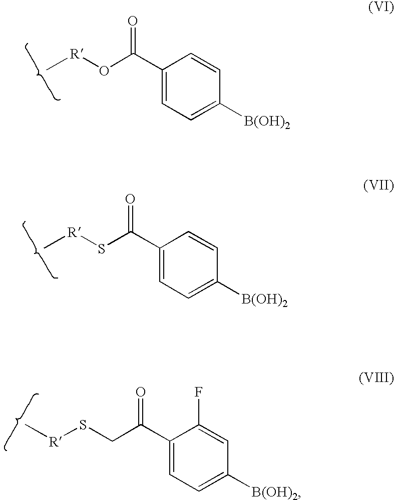
wherein R′ is a C6-C12 alkylene group.

6. The polymer of claim 1, wherein the polymer comprises polymerized monomer units represented by Structural Formula (IX), (X), or (XI): 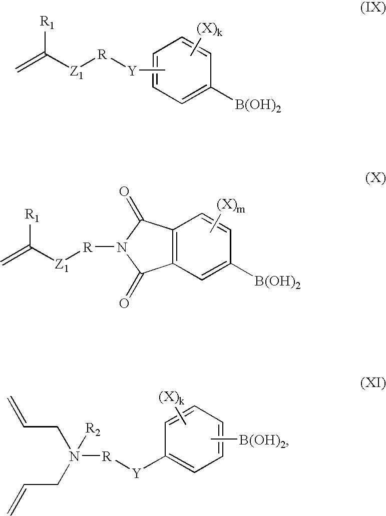
wherein:
R₁ is —H or a lower alkyl group;
R₂ is —H, a lower alkyl group, or is absent;
Z is a bond, CH₂S, S, NH, or O;
Z₁ is a bond, —C(O)NH—, —C(O)O—, —C₆H₄O—, or —C₆H₄NHC(O)—.

7. The polymer of claim 6, wherein R₁ is —H or —CH₃; each X is independently —H, a halogen, nitrile, ester or sulfone.

8. The polymer of claim 7, wherein said polymerized monomer units are represented by Structural Formula (XII) or (XIII): 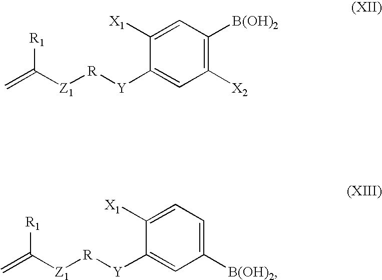
wherein X₁ and X₂ are each independently —H, a halogen or nitrile; and Y is —C(O)Z- or -ZC(O)—.

9. The polymer of claim 8, wherein R is a C6-C12 alkylene group; R₁ is —H; X₁ and X₂ are each independently —H or —F; Y is —OC(O)— or —SCH₂C(O)—; and Z₁ is —C(O)O—.

10. The polymer of claim 9, wherein the monomer units are represented by Structural Formula (XIV), (XV), (XVI), or (XVII):

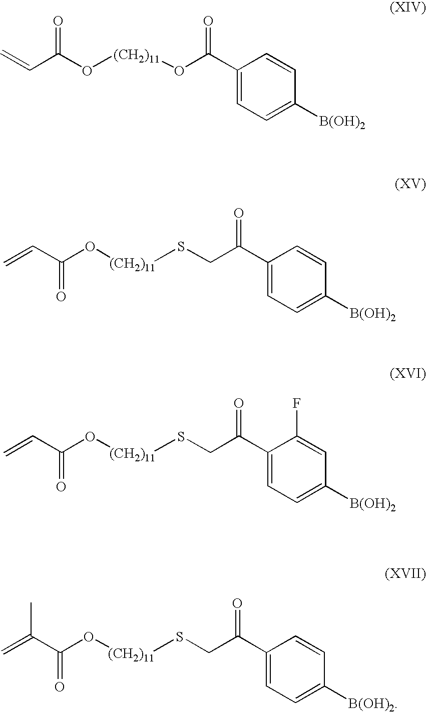

11. The polymer of claim 1, wherein said polymer is a copolymer.

12. The copolymer of claim 11, wherein said copolymer comprises a hydrophobic repeat unit.

13. The copolymer of claim 11, wherein said copolymer comprises a cationic, anionic, zwitterionic, or neutral hydrophilic repeat unit.

14. The copolymer of claim 13, wherein said copolymer comprises an anionic repeat unit or a zwitterionic repeat unit.

15. The copolymer of claim 14, wherein the anionic repeat unit or zwitterionic repeat unit comprises a sulfonic acid moiety or a salt thereof.

16. The copolymer of claim 15, wherein the anionic repeat unit is polymerized 2-acrylamido-2-methyl-1-propane sulfonic acid or a salt thereof; polymerized styrene sulfonic acid or a salt thereof; or polymerized 3-acrylatopropane sulfonic acid or a salt thereof.

17. The copolymer of claim 13, wherein said copolymer comprises a polyether sidechain.

18. The copolymer of claim 13, wherein said copolymer is a block copolymer, a graft copolymer, a comb copolymer, a star copolymer, a dendrimer, a hyperbranched polymer, or a crosslinked hydrogel.

19-23. (canceled)

24. The copolymer of claim 16, wherein the copolymer is poly{4-(14′-acryloxy-3′-thia-11′-keto)tetradecyl phenyl boronic acid-co-potassium 3-sulfopropyl acrylate)}, poly{4-(14′-methacryloxy-3′-thia-1′-keto)tetradecyl phenyl boronic acid-co-sodium 4-styrene sulfonate}, poly{11-acryloxyundecyl(4-boronato)benzoate-co-sodium 2-acrylamido-2-methyl-1-propanesulfonate}, poly{4-(14′-acryloxy-3′-thia-1′-keto)tetradecyl phenyl boronic acid-co-sodium 2-acrylamido-2-methyl-1-propanesulfonate}, or poly{4-(14′-acryloxy-3′-thia-1′-keto)tetradecyl phenyl boronic acid-co-sodium-4-styrene sulfonate}.

25. A method for treating obesity in a mammal, comprising the step of orally administering to the mammal an effective amount of a polymer substituted with at least one group represented by Structural Formula (I) or (II): 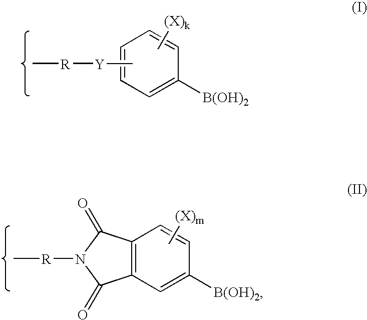
wherein:
R is a C6-C30 hydrocarbylene group optionally interrupted by one or more heteroatoms selected from the group consisting of NH, S, and O;
R₁ is —H or a lower alkyl group;
R₂ is —H, a lower alkyl group, or is absent;
each X is independently —H, a substituted or unsubstituted alkyl group, an electron withdrawing group, or an electron donating group meta to the boronic acid moiety;
Y is —C(O)Z-, -ZC(O)— or —S(CH₂)ₙ—;
Z is a bond, CH₂S, S, NH, or O;
Z₁ is a bond, —C(O)NH—, —C(O)O—, —C₆H₄O—, or —C₆H₄NHC(O)—;
m is an integer from 0 to 3;
k is an integer from 0 to 4;
n is an integer from 0 to 5.

26. The method of claim 25, further comprising the step of administering a fat binding polymer to the mammal.

27. The method of claim 25, wherein each X is independently —H, a halogen, nitrile, ester or sulfone.

28. The method of claim 27, wherein said polymer is substituted with at least one group represented by Structural Formula (V): 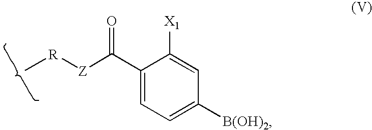
wherein X₁ is —H, a halogen, or nitrile.

29. The method of claim 28, wherein the polymer is substituted with at least one group represented by Structural Formula (VI), (VII), or (VIII): 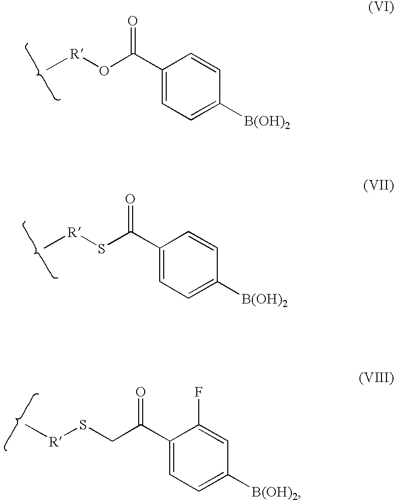
wherein R′ is a C6-C12 alkylene group.

30. The method of claim 25 wherein the polymer comprises polymerized monomer units represented by Structural Formula (IX), (X), or (XI): 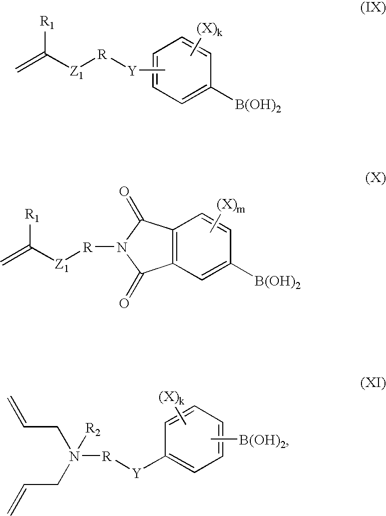
wherein:
R₁ is —H or a lower alkyl group;
R₂ is —H, a lower alkyl group, or is absent;
Z is a bond, CH₂S, S, NH, or O;
Z₁ is a bond, —C(O)NH—, —C(O)O—, —C₆H₄O—, or —C₆H₄NHC(O)—.

31. The method of claim 30, further comprising the step of administering a fat binding polymer to the mammal.

32. The method of claim 30, wherein R₁ is —H or —CH₃; and each X is independently —H, a halogen, nitrile, ester or sulfone.

33. The method of claim 32, wherein the monomer units are represented by Structural Formula (XII) or (XIII): 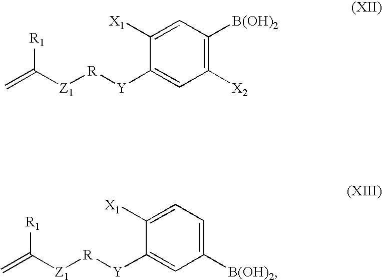
wherein X₁ and X₂ are each independently —H, a halogen or nitrile and Y is —C(O)Z- or -ZC(O)—.

34. The method of claim 33, wherein R is a C6-C12 alkylene group; R₁ is —H; X₁ and X₂ are each independently —H or —F; Y is —OC(O)— or —SCH₂C(O)—; and Z₁ is —C(O)O—.

35. The method of claim 34, wherein the monomer units are represented by Structural Formula (XIV), (XV), (XVI) or (XVII):

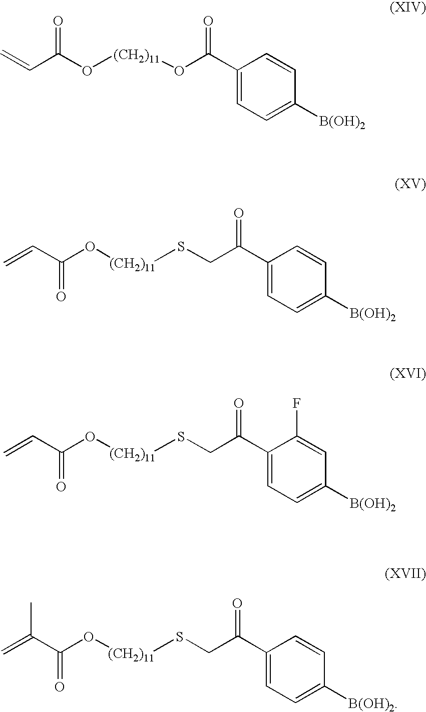

36. The method of claim 30, wherein the polymer is a copolymer.

37. The method of claim 36, wherein the copolymer comprises a hydrophobic repeat unit.

38. The method of claim 36, wherein the copolymer comprises a cationic, anionic, zwitterionic, or neutral hydrophilic repeat unit.

39. The method of claim 38, wherein the copolymer comprises an anionic repeat unit or a zwitterionic repeat unit.

40. The method of claim 39, wherein the anionic repeat unit or zwitterionic repeat unit comprises a sulfonic acid moiety or a salt thereof.

41. The method of claim 40, wherein the anionic repeat unit is polymerized 2-acrylamido-2-methyl-1-propane sulfonic acid or a salt thereof; polymerized styrene sulfonic acid or a salt thereof, or polymerized 3-acrylato-1-propane sulfonic acid or a salt thereof.

42. The method of claim 38, wherein the copolymer is a block copolymer, a graft copolymer, a comb copolymer, a star copolymer, a dendrimer, a hyperbranched polymer, or a crosslinked hydrogel.

43-47. (canceled)

48. The method of claim 41, wherein the copolymer is poly{4-(14′-acryloxy-3′-thia-1′-keto)tetradecyl phenyl boronic acid-co-potassium 3-sulfopropyl acrylate)}, poly{4-(14′-methacryloxy-3′-thia-1′-keto)tetradecyl phenyl boronic acid-co-sodium 4-styrene sulfonate}, poly{11-acryloxyundecyl(4-boronato)benzoate-co-sodium 2-acrylamido-2-methyl-1-propanesulfonate}, poly{4-(14′-acryloxy-3′-thia-1′-keto)tetradecyl phenyl boronic acid-co-sodium 2-acrylamido-2-methyl-1-propanesulfonate}, or poly{4-(14′-acryloxy-3′-thia-1′-keto)tetradecyl phenyl boronic acid-co-sodium-4-styrene sulfonate}.

49. A method for reducing absorption of fat in a mammal in need of such treatment, comprising the step of orally administering to the mammal an effective amount of a polymer substituted with at least one group represented by Structural Formula (I) or (II): 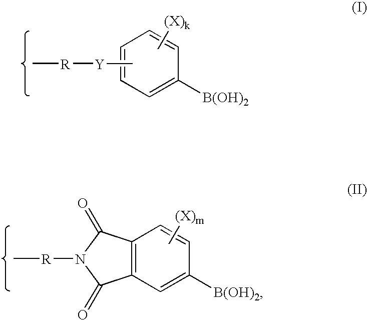
wherein:
R is a C6-C30 hydrocarbylene group optionally interrupted by one or more heteroatoms selected from the group consisting of NH, S and O;
each X is independently —H, a substituted or unsubstituted alkyl group, an electron withdrawing group, or an electron donating group meta to the boronic acid moiety;
Y is —C(O)Z-, -ZC(O)— or —S(CH₂)ₙ—;
Z is a bond, CH₂S, S, NH, or 0;
m is an integer from 0 to 3;
k is an integer from 0 to 4; and
n is an integer from 0 to 5.

50. The method of claim 49, further comprising the step of administering a fat binding polymer to the mammal.

51. The method of claim 49 wherein the polymer comprises polymerized monomer units represented by Structural Formula (IX), (X), or (XI): 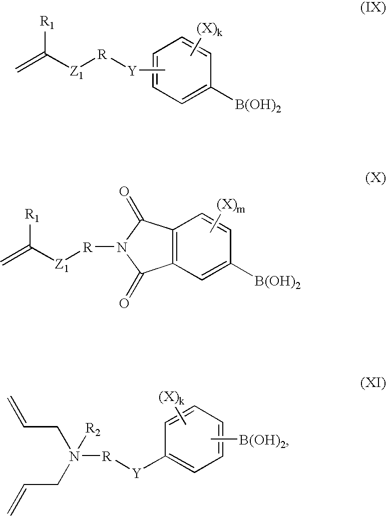
wherein:
R₁ is —H or a lower alkyl group;
R₂ is —H, a lower alkyl group, or is absent;
Z is a bond, CH₂S, S, NH, or O;
Z₁ is a bond, —C(O)NH—, —C(O)O—, —C₆H₄O—, or —C₆H₄NHC(O)—.

52. (canceled)

53. The method of claim 49, wherein the polymer is a copolymer selected from poly{4-(14′-acryloxy-3′-thia-1′-keto)tetradecyl phenyl boronic acid-co-potassium 3-sulfopropyl acrylate)}, poly{4-(14′-methacryloxy-3′-thia-1′-keto)tetradecyl phenyl boronic acid-co-sodium 4-styrene sulfonate}, poly{1-acryloxyundecyl(4-boronato)bezoate-co-sodium 2-acrylamido-2-methyl-1-propanesulfonate}, poly{4-(14′-acryloxy-3′-thia-1′-keto)tetradecyl phenyl boronic acid-co-sodium 2-acrylamido-2-methyl-1-propanesulfonate}, or poly{4-(14′-acryloxy-3′-thia-1′-keto)tetradecyl phenyl boronic acid-co-sodium-4-styrene sulfonate}.

54. The method of claim 49, wherein the subject is afflicted with one or more conditions selected from obesity, Type II diabetes mellitus, impaired glucose tolerance, hypertension, coronary thrombosis, stroke, lipid syndromes, hyperglycemia, hypertriglyceridemia, hyperlipidemia, sleep apnea, hiatal hernia, reflux esophagisitis, osteoarthritis, gout, cancers associated with weight gain, gallstones, kidney stones, pulmonary hypertension, infertility and cardiovascular disease.

55. A composition comprising the polymer of claim 1, and a pharmaceutically-acceptable carrier.
